(19) United States
(12) Patent Application Publication
(10) Pub. No.: US 2010/0240056 A1
(43) Pub. Date: Sep. 23, 2010

(54) Methods And Systems For Screening Species-Specific Insecticidal Candidates

(76) Inventor: Vincent C. Henrich, III, Greensboro, NC (US)

Correspondence Address: KILPATRICK STOCKTON LLP, 1001 WEST FOURTH STREET, WINSTON-SALEM, NC, 27101, US

(21) Appl. No.: 12/731,810

(22) Filed: Mar. 25, 2010

Related U.S. Application Data
(63) Continuation-in-part of application No. 11/543,682, filed on Oct. 5, 2006.
Application No. 11/543,682 is a continuation-in-part of application No. 10/929,090, filed on Aug. 27, 2004.
(60) Provisional application No. 61/163,392, filed on Mar. 25, 2009.
(60) Provisional application No. 60/723,724, filed on Oct. 5, 2005.
(60) Provisional application No. 60/498,847, filed on Aug. 29, 2003.

(51) Int. Cl. C12Q 1/68

(52) U.S. Cl. 435/6

(57) ABSTRACT

Disclosed are methods and systems for identifying compounds that act to modulate insect growth and/or development in a species-specific manner. Such systems and methods may provide for the identification of species-specific insecticides. The methods and systems of the invention may include at least one an isolated nucleic acid molecule that encodes a polypeptide comprising an ecdysone receptor (EcR) isoform from a first distinct species and a second isolated nucleic acid molecule comprising a DNA sequence that encodes a polypeptide comprising at least a portion of an Ultraspiracle (USP) protein from a second distinct species.

### STATEMENT OF RELATED APPLICATIONS

The present application claims priority to U.S. Provisional Patent Application No. 61/163,392, filed Mar. 25, 2009, entitled “Methods and Systems for Screening Novel Insecticide Candidates”, and is a continuation-in-part of U.S. patent application Ser. No. 11/543,682, filed Oct. 5, 2006, entitled Methods, Compositions and Systems for the Identification of Species-Specific or Developmental Stage-Specific Insecticides”, which claims priority to U.S. Provisional Patent Application 60/723,724, filed Oct. 5, 2005, and which is a continuation-in-part of U.S. patent application Ser. No. 10/929,090, filed Aug. 27, 2004 entitled “Compounds That Act To Modulate Insect Growth And Methods And Systems For Identifying Such Compounds,” which claims priority to U.S. Provisional Patent Application No. 60/498,847, filed Aug. 29, 2003. U.S. Patent Applications 61/163,392, 11/543,682, 60/723,724, 10/929,090, and 60/498,847 are each incorporated by reference herein in their entireties.

### FEDERAL SUPPORT

The work described herein was supported at least in part by Federal grants from the U.S. Department of Agriculture by a USDA CRSREES grant (2003-35302-13474). Thus, the Federal government may have rights in this invention.

### FIELD OF THE INVENTION

The present invention relates to compounds that act to modulate insect growth in a species-specific manner and methods and systems for identifying such compounds.

### BACKGROUND OF THE INVENTION

Insect development is largely driven by the action of ecdysteroids and its modulation by juvenoids. For all insects and many other arthropods, ecdysteroid action is mediated by the heterodimerization of two nuclear receptors, the ecdysone receptor (EcR) and its partner, Ultraspiracle (USP), the insect orthologue of the vertebrate retinoid X receptor (RXR). Many essential characteristics of ecdysteroid action are well-described in Drosophila melanogaster [1,2] and have since been confirmed and extended in other insect species [3,4]. Generally, one or more isoforms of EcR and USP in a given species trigger an orchestrated and multi-tiered hierarchy of transcriptional changes in target cells that ultimately mediate the morphogenetic changes associated with molting, metamorphosis, and reproductive physiology [5].

While the basic molting mechanism is highly conserved, it is apparent that the characteristics of the EcR/USP heterodimer vary among species. This is readily seen in the species-specific effects of the nonsteroidal agonists, the diacylhydrazines, which show order-specific differences in receptor affinity and in vivo toxicity [6]. Biochemical and cell culture studies of EcR and USP have also revealed species-specific functional characteristics that presumably underlie differences in ecdysteroid-driven developmental events [7-11]. Steroids and nonsteroidal agonists bind exclusively to the EcR-LBD, though the presence of USP increases ligand-binding affinity [12-15].

The diversity of ligand-responsive characteristics seen among ecdysteroid receptors from various insect species suggests a basis for identifying and screening for compounds which perturb normal receptor function [12, 13, 15, 16]. Ecdysteroid receptor-mediated transcriptional activity has been measured in mammalian cells, which have no endogenous response to insect ecdysteroids, by transfecting them with the genes encoding EcR and USP, along with an ecdysteroid-inducible reporter [17-19]. An analysis of species-specific versions of EcR and USP and site-directed mutations in this heterologous cell system has generally established that the effects of ecdysteroids and other diacylhydrazine-based agonists can be measured by reporter gene activity [8, 19, 20]. Further, the Drosophila EcR/USP heterodimer is potentiated by the presence of juvenile hormone (JH) in mammalian cells, that is, JH dramatically reduces the ecdysteroid concentration necessary to attain maximal induction from an ecdysteroid-inducible reporter gene [9, 21]. The mechanism for potentiation has not been elucidated, though it reveals a modulatory action that may be useful for identifying novel insecticides acting as disruptors of normal ecdysteroid action. This possibility heightens the importance of evaluating the heterologous cell culture assay as a valid tool for assessing ecdysteroid receptor capabilities from specific species.

Hundreds of phytocompounds which act as nonsteroidal and steroidal agonists of the insect ecdysteroid receptor have been identified [22, 23], and a large number of JH analogues and mimics have also been isolated from plants [24]. If the cell culture assay has utility as a method for detecting novel inducers and/or JH potentiators of the EcR/USP heterodimer, then receptors from an insect species such as the Colorado potato beetle (CPB), Leptinotarsa decemlineata, are expected to evoke a profile of response that varies considerably from those previously reported for Drosophila melanogaster. Further, these characteristics are expected to be consistent with in vivo measurements of ecdysteroid activity in L. decemlineata [16, 20, 25-27]. The CPB belongs to a relatively primitive insect order, the Coleoptera. Owing to its worldwide importance as a pest insect and its well-established ability to develop resistance to insecticides, the species has been well-studied for its susceptibility to a variety of agonists [28-29].

The L. decemlineata ecdysteroid receptor shows the general structural features shared by all EcR and USP sequences characterized among insects and other arthropods [5, 30-31]. Two EcR isoforms (A and B) have been identified so far in the L. decemlineata genome. The LdUSP carries a ligand-binding domain (LBD) that is remarkably similar to the vertebrate RXR, and lacks many of the features found in the fly USP, such as glycine-rich regions and a B-loop between helices 2 and 3 [30-32]. This divergence between the Coleopteran USP-LBD (often referred to as RXR) with those of Lepidoptera and Diptera has been noted, suggesting a concomitant functional divergence [32]. Thus, there is a need for methods and systems to identify and characterize candidate insecticidal compounds showing both inductive and potentiative activity.

### SUMMARY OF THE INVENTION

Embodiments of the present invention comprise nucleic acid constructs, systems, and methods for the identification of compounds that can modulate insect growth or development in a species-specific and/or developmentat stage-specific manner. In certain embodiments, the compounds may be insecticides for at least some insect species. The present invention may be embodied in a variety of ways.

In one embodiment, the present invention comprises systems for the identification of compounds that modulate insect growth and/or development in a species-specific and/or stage specific manner comprising: an isolated nucleic acid molecule that encodes a polypeptide comprising an Ecdysone receptor (EcR) isoform from a distinct species; and a second isolated nucleic acid molecule comprising a DNA sequence that encodes a polypeptide comprising at least a portion of an Ultraspiracle (USP) protein from a distinct species.

In other embodiments, the present invention comprises methods for the identification of compounds that modulate insect growth and/or development in a species-specific and/or stage specific manner comprising: (a) transfecting a first mammalian cell population with a first vector comprising a nucleotide sequence that encodes at least a portion of a USP polypeptide from a first species, and a second vector comprising a DNA sequence that encodes a polypeptide comprising an Ecdysone receptor (EcR) isoform; (b) transfecting a second mammalian cell population with a vector comprising a nucleotide sequence that encodes at least a portion of a USP polypeptide from a second species and a second vector comprising a DNA sequence that encodes for a polypeptide comprising an Ecdysone receptor (EcR) isoform; and (c) comparing the ability of a compound of interest to increase ecdysteroid-mediated transcription of the reporter gene in the first and second cell populations.

In yet other embodiments, the present invention comprises methods for the identification of compounds that modulate insect growth and/or development in a species-specific and/or stage specific manner comprising: (a) transfecting a first mammalian cell population with a first vector comprising a nucleotide sequence that encodes an Ecdysone receptor (EcR) isoform polypeptide from a first species, and a second vector comprising a DNA sequence that encodes a polypeptide comprising at least a portion of an USP polypeptide; (b) transfecting a second mammalian cell population with a vector comprising a nucleotide sequence that encodes an EcR isoform polypeptide from a second species and a second vector comprising a DNA sequence that encodes for a polypeptide comprising at least a portion of a USP polypeptide; and (c) comparing the ability of a compound of interest to increase ecdysteroid-mediated transcription of the reporter gene in the first and second cell populations.

Further details on each of these aspects of the present invention are set forth in the following description, figures, and claims. It is to be understood that the invention is not limited in its application to the details set forth in the following description, figures and claims, but is capable of other embodiments and of being practiced or carried out in various ways.

Embodiments of the present invention recognize that insect development is driven by the action of ecdysteroids on morphogenetic processes. The ecdysteroid receptor is a protein heterodimer comprised of two nuclear receptors, the ecdysone receptor (EcR) and Ultraspiracle (USP). Functional properties of EcR and USP vary among insect species and provide a basis for identifying novel and species-specific insecticidal candidates which disrupt this receptor's normal activity.

### BRIEF DESCRIPTION OF THE FIGURES

The invention may be better understood by reference to the following non-limiting figures.

FIG. 1 shows a ClustalW amino acid alignment of N-terminal (A/B) domains of EcR isoforms from Leptinotarsa decemlineata (Ld) and Drosophila melanogaster (Dm) where panel (A) shows alignment of EcR-A from DmEcRA (SEQ ID NO: 1) and LdEcRA (SEQ ID NO: 2), panel (B) shows alignment of EcR-B1 (SEQ ID NO: 3) and EcR-B2 (SEQ ID NO: 5) from D. melanogaster and EcR-B (SEQ ID NO: 4) from L. decemlineata, and panel (C) shows alignment of the most carboxy-terminal side of the A/B region shared among all isoforms of both species, for DmEcRA (SEQ ID NO: 6), LDEcRA (SEQ ID NO: 7), DmEcRB1 (SEQ ID NO: 8), DmEcRB2 (SEQ ID NO: 9) and LdEcRB (SEQ ID NO: 10).

FIG. 2 shows the effects of maximal dosages of selected agonists (20-hydroxyecdysone (20E), muristerone A (murA), makisterone A (makA), and methoxyfenozide (RH-2485) upon normalized ecdysteroid receptor-mediated transcriptional activity with sin D. melanogaster (Dm) EcR/USP (Panel A) or L. decemlineata (Ld) EcR/USP (Panel B) expressed in CHO cells in accordance with embodiments of the present invention, where all transcriptional activity values are normalized based on cell mass as measured by β-galactosidase reporter gene activity, and levels of activities were then adjusted relative to DmEcRB2/DmUSP in the absence of hormone (assigned a value of 1.0) to allow for direct comparison of quantitative transcriptional activity; data points are based on N=3, and error bars indicate one standard deviation. Panel (C) shows a Western immunoblot of Chinese Hamster Ovary (CHO) cell extracts expressing the EcR vectors described herein as detected with 9B9 monoclonal antibody in accordance with embodiments of the present invention wherein extracts from cells grown in culture medium with no added agonist were equalized for gel loading based on β-galactosidase reporter gene activity; densitometry readings for individual signals were adjusted relative to DmEcR-B2 (equals 1.0).

FIG. 3 shows fold induction of natural ecdysteroids, 20-hydroxyecdysone (20E), muristerone A (murA), ponasterone A (ponA), and makisterone A (makA) upon ecdysteroid receptor-mediated transcriptional activity in CHO cells over a dosage range in accordance with embodiments of the present invention where panel (A) shows stimulation of D. melanogaster EcRB2/VP16-DmUSP, and panel (B) shows L. decemlineata EcRA/VP16-LdUSP, and panel (C) shows L. decemlineata EcRB/VP16-LdUSP, wherein all luciferase activity levels were normalized based on β-galactosidase activity as a measure of cell mass, and for each agonist, fold-inductions are shown relative to the normalized luciferase activity observed in the absence of the test agonist (assigned a value of 1); all data points are based on N=3 that were tested at the same time and error bars indicate one standard deviation.

FIG. 4 shows fold induction of nonsteroidal agonists, halofenozide (RH-0345), methoxyfenozide (RH-2485), RH-5849 and tebufenozide (RH-5992) upon ecdysteroid receptor-mediated transcriptional activity in CHO cells over a dosage range in accordance with embodiments of the present invention where panel (A) shows D. melanogaster EcR-B2/VP16-DmUSP, panel (B) shows L. decemlineata EcR-A/VP16-LdUSP, and panel (C) shows L. decemlineata EcRB/VP16-LdUSP, wherein all luciferase activity levels were normalized based on β-galactosidase activity as a measure of cell mass, and for each agonist, fold-inductions are shown relative to the normalized luciferase activity observed in the absence of the test agonist (assigned a value of 1); all data points are based on N=3 that were tested at the same time, and error bars indicate one standard deviation.

FIG. 5 shows an electrophoretic mobility shift assay (EMSA) using CHO cell extracts following transfection and incubation in the absence and presence of murA, RH-5849 and RH-5992 (tebufenozide), using the hsp27 EcRE as a labeled probe in accordance with alternate embodiments of the present invention, wherein an asterisk (*) designates a shift band, and all extracts were equilibrated by β-galactosidase activity prior to loading; densitometry readings corresponding to designated shift bands are indicated below the image and adjusted relative to the signal generated by LdEcR-B (equals 1.0).

FIG. 6 shows effects of juvenile hormone III (JHIII) on transcriptional activity induced by muristerone A (murA) (Panel A) and RH-2485 (Panel B) for D. melanogaster EcR-B2/VP16-DmUSP and analogous LdEcR/VP16-LdUSP complexes in accordance with embodiments of the present invention where the parentheses in panel A indicate a potentiation effect, and the arrows in panel B indicate absence of potentiation when RH-2485 is the agonist; all transcriptional activity levels were adjusted to DmEcR-B2/VP16-DmUSP in the absence of ligand (assigned a value of 1.0), and no effect upon transcriptional activity was observed when JH-III was tested with RH2485 (not shown).

FIG. 7 shows effects of VP16-USP and VP-16 USP/Δ DBD on muristerone A(murA)-inducible transcriptional activity at 2.5 μM muristone in accordance with embodiments of the present invention wherein panel (A) shows Drosophila melanogaster (Dm) EcR-B1 and EcR-B2 with VP16DmUSP and VP16-DmUSP/ΔDBD, panel (B) shows Leptinotarsa decemlineata (Ld) EcR-A and EcR-B with VP16LdUSP and VP16-LdUSP/ΔDBD, and panel (C) shows results with cross-species EcR/USP heterodimers; all levels were adjusted to the activity observed in EcRB2/VP16-DmUSP in the absence of agonist (equals 1.0), with all data points based on N=3 and were run simultaneously and error bars indicate one standard deviation.

### DETAILED DESCRIPTION

Full citations for references referred to herein by number are provided in the reference section.

The term “a” or “an” as used herein may refer to more than one object unless the context clearly indicates otherwise. The term “or” is used interchangeably with the term “and/or” unless the context clearly indicates otherwise.

As used herein, “stage-specific” or “developmental stage-specific” insecticides are insecticides that display increased toxicity at one stage of insect development as compared to other stages of insect development. As used herein, “species-specific” insecticides are insecticides that display increased toxicity for a particular species of insect as compared to other species of insects.

As used herein, a “ligand” is a molecule that interact either directly or indirectly with a receptor to form a complex. An “agonist” comprises a compound that binds to a receptor to form a complex that elicits a pharmacological response specific to the receptor involved.

An “antagonist” comprises a compound that binds to an agonist or a receptor to form a complex that does not give rise to a substantial pharmacological response and can inhibit the biological response induced by an agonist.

“Polypeptide” and “protein” are used interchangeably herein to describe protein molecules that may comprise either partial or full-length proteins. As used herein, a “polypeptide domain” comprises a region along a polypeptide that comprises an independent unit. Domains may be defined in terms of structure, sequence and/or biological activity. In one embodiment, a polypeptide domain may comprise a region of a protein that folds in a manner that is substantially independent from the rest of the protein. Domains may be identified using domain databases such as, but not limited to PFAM, PRODOM, PROSITE, BLOCKS, PRINTS, SBASE, ISREC PROFILES, SAMRT, and PROCLASS.

A “nucleic acid” is a polynucleotide such as deoxyribonucleic acid (DNA) or ribonucleic acid (RNA). The term is used to include single-stranded nucleic acids, double-stranded nucleic acids, and RNA and DNA made from nucleotide or nucleoside analogues.

The term “vector” refers to a nucleic acid molecule that may be used to transport a second nucleic acid molecule into a cell. In one embodiment, the vector allows for replication of DNA sequences inserted into the vector. The vector may comprise a promoter to enhance expression of the nucleic acid molecule in at least some host cells. Vectors may replicate autonomously (extrachromasomal) or may be integrated into a host cell chromosome. In one embodiment, the vector may comprise an expression vector capable of producing a protein derived from at least part of a nucleic acid sequence inserted into the vector.

The term “fusion protein” may refer to a protein or polypeptide that has an amino acid sequence derived from two or more proteins. The fusion protein may also include linking regions of amino acids between amino acids portions derived from separate proteins. Unless specifically stated, there is no required order of linking polypeptides to form a fusion protein.

The term “percent identical” or “percent identity” refers to sequence identity between two amino acid sequences or between two nucleic acid sequences. Percent identity can be determined by aligning two sequences and refers to the number of identical residues (i.e., amino acid or nucleotide) at positions shared by the compared sequences. Sequence alignment and comparison may be conducted using the algorithms standard in the art (e.g. Smith and Waterman, Adv. Appl. Math., 1981, 2:482; Needleman and Wunsch, 1970, J. Mol. Biol., 48:443); Pearson and Lipman, 1988, Proc. Natl. Acad. Sci. USA, 85:2444) or by computerized versions of these algorithms (Wisconsin Genetics Software Package Release 7.0, Genetics Computer Group, 575 Science Drive, Madison, Wis.) publicly available as BLAST and FASTA. Also, ENTREZ, available through the National Institutes of Health, Bethesda Md., may be used for sequence comparison. In one embodiment, percent identity of two sequences may be determined using GCG with a gap weight of 1, such that each amino acid gap is weighted as if it were a single amino acid or nucleotide mismatch between the two sequences.

An “effective amount” as used herein means the amount of an agent that is effective for producing a desired effect. Where the agent is being used to achieve a insecticidal effect, the actual dose which comprises the effective amount may depend upon the route of administration, and the formulation being used.

As used herein, “modulation of insect growth” includes the modulation of the growth of an individual insect or an insect population and includes modulation of insect reproduction, morphogenesis, and survival. As used herein, “insect growth” comprises growth and development of an individual insect and/or an insect population and thus, refers to the growth, morphogenesis, and survival of an individual insect, or an insect population.

As used herein, an “increase in transcription” comprises an increase from a measurable basal level to a higher level. Alternatively, an “increase in transcription” may comprise an increase from an undetectable level to a measurable level. As used herein, “activation of EcR” describes increasing EcR-mediated transcription. As used herein, “EcR-mediated transcription” comprises a gene transcription event that requires binding of the activated ecdysone receptor to a promoter upstream of the gene being transcribed.

As used herein, “potentiation” of agonist-activated transcription comprises an increase in transcription induced by compounds that do not increase EcR-mediated transcription when added alone, but that can increase agonist-activated transcription at sub-maximal levels of the agonist.

A “hormone response element” (HRE) comprises a nucleotide region upstream of a gene that mediates the effect of a steroid hormone. An “isoform” is a variant form of a protein that has the same general function as another protein but which may have small differences in its sequence either because it is encoded by a different gene, is expressed by a different promoter in the same gene, or is derived by alternative splicing of the same pre-mRNA. For example, EcR may exists in at least three versions having trans-activating regions that differ in sequence to provide isoforms EcRA, EcRB, and EcRB2, each of which can activate transcription of a gene having an EcR HRE in its promoter. EcRA is derived from a different promoter of the EcR gene, and B1 and B2 are derived from alternative splicing of a pre-mRNA.

A “ligand binding domain” is that portion of a protein or polypeptide involved in binding of a ligand.

A “juvenile hormone mimetic” is a compound that functions like any one of the natural juvenile hormones such as JHI, JH II, or JH III. The normal physiological functions of the naturally-occurring compounds are compromised by ectopic or exogenous administration of any number of juvenile hormone mimetic compounds.

An “insecticide synergist” is a compound that acts in synergy with an insecticide to provide a response that is greater than additive.

Embodiments of the present invention comprise nucleic acid constructs and systems comprising such constructs for the identification of compounds that modulate insect growth and/or development in a species-specific and/or stage specific manner and methods of using such constructs and/or systems. In certain embodiments, the compounds may be insecticides for at least some insect species. The present invention may be embodied in a variety of ways.

In one embodiment, the present invention comprises a system for the identification of compounds that modulate insect growth and/or development in a species-specific and/or stage specific manner comprising: an isolated nucleic acid molecule that encodes a polypeptide comprising an Ecdysone receptor (EcR) isoform from a distinct species; and a second isolated nucleic acid molecule comprising a DNA sequence that encodes a polypeptide comprising at least a portion of an Ultraspiracle (USP) protein from a distinct species. In certain embodiments, the compounds comprise species-specific insecticides

In one embodiment, the system may comprise an isolated nucleic acid molecule that encodes a polypeptide comprising an ecdysone receptor (EcR) isoform from a first species and an isolated nucleic acid molecule comprising a DNA sequence that encodes a polypeptide comprising at least a portion of an Ultraspiracle (USP) protein from a second species.

In certain embodiments, the system is a biological system. For example, the system may comprise a cell or a population of cells transfected with the isolated DNA sequences of the system. In one embodiment, the system may comprise a cell or a population of cells transfected with an isolated nucleic acid that encodes a polypeptide comprising an ecdysone receptor (EcR) isoform from a first species and a nucleic acid molecule comprising a DNA sequence that encodes a polypeptide comprising at least a portion of an Ultraspiracle (USP) protein from the first (i.e., the same) species and another cell or population of cells transfected with an isolated nucleic acid molecule that encodes a polypeptide comprising an ecdysone receptor (EcR) isoform from a second species and a nucleic acid molecule comprising a DNA sequence that encodes a polypeptide comprising at least a portion of an Ultraspiracle (USP) protein from the second species. Or, the system may comprise a cell or a population of cells transfected with an isolated nucleic acid that encodes a polypeptide comprising an ecdysone receptor (EcR) isoform from a first species and nucleic acid molecule comprising a DNA sequence that encodes a polypeptide comprising at least a portion of an Ultraspiracle (USP) protein from the second species. In certain embodiments, the system may further comprise a second cell or population of cells transfected with an isolated nucleic acid that encodes a polypeptide comprising an ecdysone receptor (EcR) isoform from a second species and nucleic acid molecule comprising a DNA sequence that encodes a polypeptide comprising at least a portion of an Ultraspiracle (USP) protein from the first species.

In yet other embodiments, additional cells or populations of cells may be transfected with EcR and/or USP proteins from third, fourth, fifth and additional species such that various species may be compared and/or utilized in combinations. For example, in one non-limiting embodiment, the system may comprise a cell or a population of cells transfected with an isolated nucleic acid that encodes a polypeptide comprising an ecdysone receptor (EcR) isoform from a first species and a nucleic acid molecule comprising a DNA sequence that encodes a polypeptide comprising at least a portion of an Ultraspiracle (USP) protein from the first (i.e., the same) species and another cell or population of cells transfected with an isolated nucleic acid molecule that encodes a polypeptide comprising an ecdysone receptor (EcR) isoform from a second species and a nucleic acid molecule comprising a DNA sequence that encodes a polypeptide comprising at least a portion of an Ultraspiracle (USP) protein from the second species, and another cell or population of cells transfected with an isolated nucleic acid molecule that encodes a polypeptide comprising an ecdysone receptor (EcR) isoform from a third species and a nucleic acid molecule comprising a DNA sequence that encodes a polypeptide comprising at least a portion of an Ultraspiracle (USP) protein from the third species, and another cell or population of cells transfected with an isolated nucleic acid molecule that encodes a polypeptide comprising an ecdysone receptor (EcR) isoform from a fourth species and a nucleic acid molecule comprising a DNA sequence that encodes a polypeptide comprising at least a portion of an Ultraspiracle (USP) protein from the fourth species, and so on such that constructs from multiple species may be evaluated. Also, the system may comprise a cell or a population of cells transfected with an isolated nucleic acid that encodes a polypeptide comprising an ecdysone receptor (EcR) isoform from a first species and nucleic acid molecule comprising a DNA sequence that encodes a polypeptide comprising at least a portion of an Ultraspiracle (USP) protein from a second species and/or a third species and/or a fourth species, and/or a fifth, sixth, seventh and additional species. In certain embodiments, the system may further comprise a second cell or population of cells transfected with an isolated nucleic acid that encodes a polypeptide comprising an ecdysone receptor (EcR) isoform from a second, and/or third, and/or fourth, and/or fifth, and/or sixth and additional species and nucleic acid molecule comprising a DNA sequence that encodes a polypeptide comprising at least a portion of an Ultraspiracle (USP) protein from the first species.

In other embodiments, the present invention comprises methods for identifying compounds that modulate insect growth and/or development in a species-specific and/or stage specific manner. In certain embodiments, the compounds may be insecticides for at least some insect species.

For example, in certain embodiments, the present invention provides a method for the identification of compounds that modulate insect growth and/or development in a species-specific and/or stage specific manner comprising: (a) transfecting a first mammalian cell or cell population with a first vector comprising a nucleotide sequence that encodes at least a portion of a USP polypeptide, and a second vector comprising a DNA sequence that encodes a polypeptide comprising an ecdysone receptor (EcR) isoform; (b) transfecting a second mammalian cell or cell population with a vector comprising a nucleotide sequence that encodes at least a portion of a USP polypeptide and a second vector comprising a DNA sequence that encodes for a polypeptide comprising an ecdysone receptor (EcR) isoform; and (c) comparing the ability of a compound of interest to increase ecdysteroid-mediated transcription of the reporter gene in the first and second cells populations of cells.

In an embodiment, the EcR isoform and the USP of step (a) are each from a first species and the EcR isoform and the USP of step (b) are each from a second species. Alternatively, at least one of the EcR isoform or the USP of step (a) are from a first species and at least one of the EcR isoform or the USP of step (b) are each from a second species. Or the USP of step (a) and step (b) may each be from the same species. Or, the USP of step (a) and step (b) may each be from different species. Additionally or alternatively, the EcR of step (a) and step (b) may be from the same species. Or, the EcR of step (a) and step (b) may be from different species.

For example, the present invention may comprise a method for the identification of compounds that modulate insect growth and/or development in a species-specific and/or stage specific manner comprising: (a) transfecting a first mammalian cell or cell population with a first vector comprising a nucleotide sequence that encodes at least a portion of a USP polypeptide from a first species, and a second vector comprising a DNA sequence that encodes a polypeptide comprising an Ecdysone receptor (EcR) isoform; (b) transfecting a second mammalian cell or cell population with a vector comprising a nucleotide sequence that encodes at least a portion of a USP polypeptide from a second species and a second vector comprising a DNA sequence that encodes for a polypeptide comprising an Ecdysone receptor (EcR) isoform; and (c) comparing the ability of a compound of interest to increase ecdysteroid-mediated transcription of the reporter gene in the first and second cells or cell populations. In alternate embodiments, the species used for the EcR and USP of step (a) are the same or different. Also, in alternate embodiments, the species used for the EcR and USP of step (b) are the same or different.

In another embodiment, the present invention comprises a method for the identification of compounds that modulate insect growth and/or development in a species-specific and/or stage specific manner comprising: (a) transfecting a first mammalian cell or cell population with a first vector comprising a nucleotide sequence that encodes an Ecdysone receptor (EcR) isoform polypeptide from a first species, and a second vector comprising a DNA sequence that encodes a polypeptide comprising at least a portion of a USP polypeptide; (b) transfecting a second mammalian cell or cell population with a vector comprising a nucleotide sequence that encodes an Ecdysone receptor (EcR) isoform from a second species and a second vector comprising a DNA sequence that encodes for a polypeptide comprising at least a portion of a USP polypeptide; and (c) comparing the ability of a compound of interest to increase ecdysteroid-mediated transcription of the reporter gene in the first and second cells or cell populations. In alternate embodiments, the species used for the EcR and USP of step (a) are the same or different. Also, in alternate embodiments, the species used for the EcR and USP of step (b) are the same or different.

Also, as described above, for the system, the methods may comprise comparing various combinations of EcR isoforms (e.g., from a second, and/or third, and/or fourth, and/or fifth, and/or sixth and/or additional species) with a USP from the first species, or comparing various combinations of USP proteins (e.g., from a second, third, fourth, fifth, sixth and additional species) with an EcR isoform from a first species. In each of the embodiments of the methods of the present invention the identified compounds may be insecticides for at least some insect species.

A variety of insect species may be used in both the methods and the systems of the present invention. In certain embodiments, the species may comprise at least one of Drosophila melanogaster, Leptinotarsa decemlineata, Chroristoneura fumerifana, Manduca sexta, Locusta migratoria, Heliothis virescens, Apis mellifera, Aedes aegypti, and Tenebrio molitor.

Thus, in alternate embodiments, the portion of the EcR or USP polypeptide used in the constructs, methods and/or systems of the present invention may be derived from an insects. Any insect comprising a EcR and/or USP protein or analogous proteins may be used for the methods of the present invention. In an embodiment, the insect may comprise at least one of a diptheria, hemiptera, coleoptera, neuroptera, lepitdoptera, or ants. In certain embodiments, the insect species comprises at least one of aphids, scale insects, leaf hoppers, white fly and blowflies. For example the insect species may comprise insects that are found on alfalfa, such as the alfalfa caterpillar (e.g., Colias eurytheme), Alfalfa Weevil (e.g., Hypera postica) Beet Armyworm (e.g., Spodoptera exigua), Blister Beetles (e.g., Epicauta spp., Lytta spp.), Soybean aphid (Aphis glycines), Blue Alfalfa Aphid (e.g., Acrythosiphon kondoi) and Pea Aphid (e.g., Acrythosiphon pisum), Cowpea Aphid (e.g., Aphis craccivora), Egyption Alfalfa Weevil (e.g., Hypera brunneipennis), Alfalfa Grasshoppers (e.g., Melanoplus spp; Trimerotropis spp), Alphafa Leafhoppers such as the Garden Leafhopper (e.g., Empoasca solana), Potato Leafhopper (e.g., E. fabae), Mexican Leafhopper (e.g., E. mexara), the Alphafa Mormon Cricket (e.g., Anabrus simplex), Spider Mits (e.g., Tetranychus urticae, Tetranychus spp.), Spottted Alfalfa Aphid (e.g., Therioaphis maculata), Variegated and Other Cutworms such as the Granulate cutworm (e.g., Agrotis subterranea), Variegated cutworm (e.g., Peridroma saucia), the Webworm (e.g., Loxosteg spp.), or the Western Yellowstriped Armyworm (e.g., Spodoptera praefica). Or, the insect species may comprise insects that are found on apples such as the Apple Magot (e.g., Rhagoletis pomonella), Apple Pandemis (e.g., Pandemis pyrusana), Caodling Moth (e.g, Cydia pomonella), Cribrate Weevil (e.g., Otiorhynchus cibricollis), European Red Mite (e.g., Panonychus ulmi) Eyespotted Bud Moth (e.g., Spilonota ocellana), Fruittre Leafroller (e.g., Archips argyrospila), Green Apple Aphid (e.g., Aphis pomi), Green Fruitworms such as the Speckled Green Fruitworm (e.g., Orthosia hibisci), Humped Green Fruitworm (e.g., Amphipyra pyramidoides), Leafhoppers such as the white apple leafhopper (e.g., Typhlocyba pomaria) or the Rose Leafhopper (e.g., Edwadsiana rosae), Leafminers (e.g., Phyllonorycter spp.), Lygus Bugs (e.g., Lygus hesperus; Lygus ellisus), Obliquebanded Leafroller (e.g., Choristoneura rosaceana), Omnivorous Leafroller, Orange Tortrix, Rosy Apple Aphid, and the like. Or the insect species may comprise insects that are found on small grains such as Armyworms (e.g, Pseudaletia unipuncta) and the Western Yellowstriped Armyworm (e.g., Spodoptera praefica), Bird Cherry-oat Aphid (e.g., Rhopalosiphum padi), Black Grass Bug (e.g., Irbisia spp), Corn Leaf Aphid (e.g., Rhopalsiphum maidis), Greenbug (e.g., Schizaphis graminum), Range Crane Fly (e.g., Tipula spp), Russian Wheat Aphid (e.g., Diuraphis noxia), Wheat Stem Maggot (e.g., Meromyza americana), Wireworms (e.g., Aeolus sp., Anchastus spp. Melanotus spp., Limonius spp.), Potato Tuberworm (e.g., Phthorimaea operculella), Flea Beetles (e.g., Epitrix spp), or Siverleaf Whitefly (e.g., Bemisia argentifolii). Or, the insect species may comprise insects that are found on corn such as Armyworms (e.g., Pseudaletia unipuncta), the Western Yellowstriped Armyworm (e.g., Spodoptera praefica), or the Beet Armyworm (e.g., Spodoptera exigua), Corn Earworm (e.g., Helicoverpa zea), Corn Leafhopper (e.g., Dalbulus maidis), Corn Leafminer (e.g., Agromyza sp.), Cucumber Beetles such as the Western Spotted Cucumber Beetle (e.g., Acalymma trivittatum), the Banded Cucumber Beetle (e.g., Diabrotica Balteata), the Spotted Cumber Beetle (e.g., Diabrotica undecimpunctata howardi), the Seedcorn Maggot (e.g., Delia platura), Thrips (e.g., Frankliniella occidentalis, Frankliniella williamsi), or False Church Bug (Nysius raphanus) and the like. Or insects that are found on celery, such as the Omnivorous leafroller (e.g., Platynota stultana), False celery Leafteri (e.g., Udea profundalis). Additional insects may comprise insects found on strawberries such as the Cycalmen Mite (e.g., Phytonemus pallidus), European Earwig (e.g., Forficula auricularia), Garden Symphylan (Scutigerella immaculata), or Root Beetles such as the Black vine weevil (e.g., Otiorhynchus sulcatus), Crbrate weevil (Otiorhynchus cribricollis), Fuller rose weevil (e.g., Pantomorus cervinus), Woods weevil (e.g., Nemocetes incomptus), or the Hoplia beetle (e.g, Hoplia dispar; H. callipyge). Or the insect may comprise a Pink Bollworm (e.g., Pectinophora gossypiella), Saltmarsh Catterpillar (e.g., Estigmene acrea), or Tabacco Budworm (e.g., Heliothis virescens). Or the insect may comprise a slug such as the Garden slug (e.g., Anion hortensis), Little Gray Slug (e.g., Deroeras reticulatum). Or the insect may comprise an insect found in trees such as the Hemlock Woolly Adelgid (e.g., Adelges tsugae), Pine Bark Beetle, and the like.

Both the methods and the systems of the present invention may comprise various types of USP molecules. In certain embodiments the nucleic acid sequence that encodes the USP polypeptide comprises a sequence that encodes: (a) a mammalian nuclear receptor transactivation domain; and/or (b) a hinge region and a ligand binding domain of an insect USP protein. The USP polypeptide may, in certain embodiments, include a sequence that encodes the DNA binding domain of the USP protein. Or, the DNA binding domain may be deleted at least in part; such constructs are designated as ΔDBD.

The methods and systems of the present invention may comprise a means to quantify expression from a EcR-USP interaction. For example, in certain embodiments, the methods and systems may further comprise transfection of the cell or population of cells with an isolated nucleic acid sequence comprising a reporter gene operably linked to a hormone response element. The reporter gene may comprise chloramphenical acetyltransferase (CAT), luciferase (LUC) or other reporter genes known in the art.

A variety of species-specific and/or stage-specific EcR and/or USP constructs may be used in the methods and systems of the present invention. For example, in certain embodiments, at least one of the isolated nucleic acid molecules is one of the following Drosophila melanogaster EcR isoforms: EcRA, EcRB1, and EcRB2. Additionally or alternatively, at least one of the isolated nucleic acid molecules is one of the following Leptinotarsa decemlineata EcR isoforms: EcRA and EcRB. Additionally or alternatively, at least one of the isolated nucleic acid molecules is a VP 16-Leptinotarsa decemlineata USP (VP16-LdUSP). Additionally or alternatively, at least one of the isolated nucleic acid molecules is a VP 16-Drosophila melanogaster USP (VP16-DmUSP). Or, other constructs may be employed.

A variety of compounds may be tested as either agonists or potentiators using the systems or methods of the present invention. For example, in certain embodiments, the agonist is an EcR agonist. In certain embodiments, the agonist comprises muristerone A, Ponasterone A, makisterone A, RH2485, RH5849, RH5992, or RH0345.

In yet other embodiments, the present invention comprises a cell or cell population transfected with one of the constructs described herein. In certain embodiments, the cell or cell population is a mammalian cell or cell population such as, but not limited to, a CHO cell.

In certain embodiments, the systems or methods of the present invention may comprise a compound to be tested for its ability to potentiate EcR-mediated transcription by an ecdysteroid agonist.

As noted above, in certain embodiments, the present invention comprises a method for the identification of species-specific and stage-specific insecticides comprising: (a) transfecting a first mammalian cell population with a first vector comprising a nucleotide sequence that encodes at least a portion of a USP polypeptide, and a second vector comprising a DNA sequence that encodes a polypeptide comprising an Ecdysone receptor (EcR) isoform; (b) transfecting a second mammalian cell population with a vector comprising a nucleotide sequence that encodes at least a portion of a USP polypeptide from the same or a different species than the USP of (a) and a second vector comprising a DNA sequence that encodes for a polypeptide comprising an Ecdysone receptor (EcR) isoform from the same or a different species than the USP of (a); and (c) comparing the ability of a compound of interest to increase ecdysteroid-mediated transcription of the reporter gene in the first and second cell populations. In alternate embodiments, the species used for the EcR and USP of step (a) are the same or different. Also, in alternate embodiments, the species used for the EcR and USP of step (b) are the same or different.

Thus, a variety of combinations of EcR and/or USP polypeptides may be used. For example, in certain embodiments, the second vector of step (a) comprises a DNA sequence that encodes a polypeptide comprising an ecdysone receptor (EcR) isoform that encodes for a polypeptide comprising an ecdysone receptor (EcR) isoform from the first species. Additionally or alternatively, the second vector of step (a) comprises a DNA sequence that encodes a polypeptide comprising an ecdysone receptor (EcR) isoform that encodes for a polypeptide comprising an ecdysone receptor (EcR) isoform from the second species. Additionally or alternatively, the second vector of step (b) comprises a DNA sequence that encodes a polypeptide comprising an Ecdysone receptor (EcR) isoform that encodes for a polypeptide comprising an Ecdysone receptor (EcR) isoform from a second species. Additionally or alternatively, the second vector of step (b) comprises a DNA sequence that encodes a polypeptide comprising an Ecdysone receptor (EcR) isoform that encodes for a polypeptide comprising an Ecdysone receptor (EcR) isoform from the first species.

In another embodiment, the present invention comprises a method for the identification of species-specific and stage-specific insecticides comprising: (a) transfecting a first mammalian cell population with a first vector comprising a nucleotide sequence that encodes an Ecdysone receptor (EcR) isoform polypeptide from a first species, and a second vector comprising a DNA sequence that encodes a polypeptide comprising at least a portion of a USP polypeptide; (b) transfecting a second mammalian cell population with a vector comprising a nucleotide sequence that encodes an Ecdysone receptor (EcR) isoform from a second species and a second vector comprising a DNA sequence that encodes for at least a portion of a polypeptide comprising a USP polypeptide; and (c) comparing the ability of a compound of interest to increase ecdysteroid-mediated transcription of the reporter gene in the first and second cell populations. In alternate embodiments, the species used for the EcR and USP of step (a) are the same or different. Also, in alternate embodiments, the species used for the EcR and USP of step (b) are the same or different.

A variety of combinations of EcR and/or USP polypeptides may be used. For example, in certain embodiments, the second vector of step (a) comprises a DNA sequence that encodes a polypeptide comprising a UPS polypeptide from the first species. Additionally or alternatively, the second vector of step (a) comprises a DNA sequence that encodes a polypeptide comprising a USP polypeptide from the second species. Additionally or alternatively, the second vector of step (b) comprises a DNA sequence that encodes a USP polypeptide from the second species. Additionally or alternatively, the second vector of step (b) comprises a DNA sequence that encodes a USP polypeptide from the first species.

In certain embodiments, the systems or methods of the present invention may comprise a compound to be tested for its ability to potentiate EcR-mediated transcription by an ecdysteroid agonist.

Thus, in certain embodiments, the methods and or system may further comprise adding a compound to be tested for its ability to potentiate EcR-mediated transcription by an EcR agonist and measuring whether there is an increase in EcR-mediated transcription when the compound to be tested is added with the EcR agonist, as compared to when only the agonist is added, or when only the compound to be tested for its ability to potentiate EcR-mediated transcription is added, such that a compound that increases ecdysteroid-activated EcR-mediated transcription to a maximum level at agonist concentrations that are normally sub-maximal, but does not directly increase EcR-mediated transcription, is a potentiator compound.

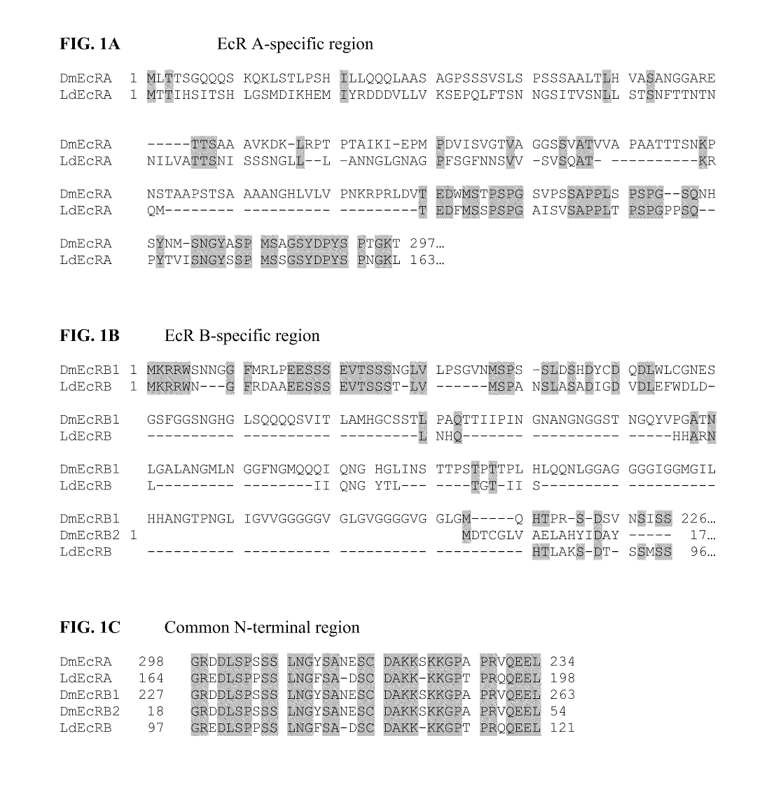

Thus, embodiments of the present invention recognize that whereas the DNA binding domains (DBDs) of EcR and USP receptors are highly similar, there is sequence specificity in the ligand binding domains (LBDs). For example, the DBDs of the Leptinotarsa and Drosophila EcR and USP receptors are identical at every amino acid position that is conserved among all EcR and USP DBD sequences respectively, and share an overall identity of over 90% in both cases [31]. Thus, in an embodiment, the canonical hsp27 EcRE, can allow direct comparisons of agonist inducibility when tested with EcR/USP from each of the two species. However, as reported elsewhere, and as shown in FIG. 1, sequence conservation is not as extensively shared in the LBD, where the identity between Drosophila and Leptinotarsa EcR is about 67% [21], and the USP LBD conservation is less than 39% between the two species [21]. In an embodiment, this divergence in the LBDs of EcR and USP between the two species provides different ligand-dependent transcriptional capabilities (AF2).

Also, the N-terminal (A/B) domains of EcR are divergent in various insect species [e.g., 31] although all of the isoforms from at least some species, e.g., Drosophila and Leptinotarsa, may share identity over a stretch amino acids that lie just to the N-terminal side of the DBD (see e.g., FIG. 1C). Interestingly, although the EcR-A isoforms from the two species share a few similar motifs in the middle region of the A/B domain (FIG. 1A), the LdEcR-B shares some identity with DmEcRB1 only in the most N-terminal region (FIG. 1B). DmEcR-B2 is unique except in the aforementioned common region shared by all the isoforms (FIGS. 1B and 1C). Thus, in certain embodiments, the divergence of sequences in the EcR A/B domain, where ligand-independent transcriptional activity (AF1) resides, may lead to species-specific transcriptional activity.

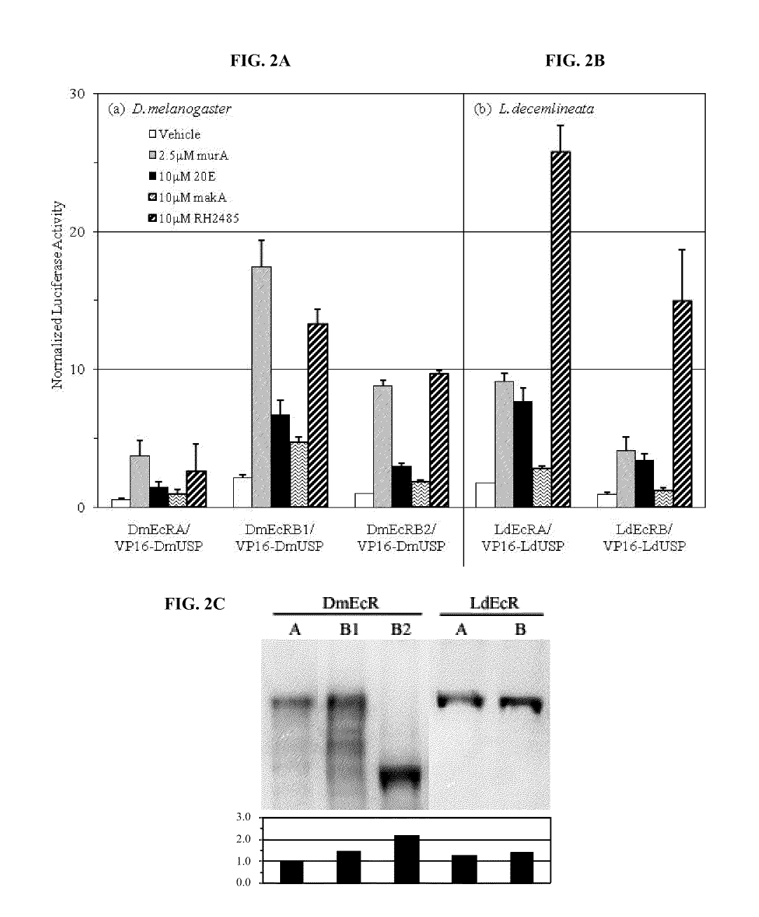

In an embodiment, the basal and ligand-induced properties of the EcR isoforms from a first species, e.g., the three D. melanogaster isoforms (DmEcR-A, DmEcR-B1 and DmEcR-B2) with the VP16-DmUSP heterodimer, may be compared with a second species, e.g., those of the L. decemlineata isoforms (e.g., EcR-A and EcR-B). As shown in FIG. 2, in certain embodiments, the EcR isoforms from the first and second species may be paired with VP16-USP constructs (i.e., the VP16-DmUSP construct and the equivalent VP16-LdUSP construct). In this embodiment of the system of the present invention, the VP16 activation domain replaces the highly variable N-terminal domain (A/B) of USP from both species, allowing for direct cross-species comparison of activity [18]. Activity may be determined by measuring reporter gene (luciferase) activity mediated by the hsp27 EcRE.

In an embodiment, to compare the efficacy of agonists, maximally-inducing dosages of several ecdysteroids and the most inductive nonsteroidal agonist, RH2485, based on preliminary experiments, may be evaluated.

In an embodiment, the pattern of response to various agonists may be similar for each of the three D. melanogaster isoforms (FIG. 2A). For example, as shown in FIG. 2A testing maximal doses of agonists for stimulation of EcR-mediated transcription, murA may evoke the strongest fold-induction, and the greatest absolute level of transcriptional activity, with RH-2485 also evoking a response from all three Drosophila EcR isoforms, and lesser responses from the natural molting hormone, 20E, and makisterone A. Also, as noted previously [9], differences in the quantitative levels of transcription may be found, with Drosophila EcR-B1 showing the highest levels of basal and induced activity, and EcR-A displaying the lowest levels of activity.

In an embodiment, the systems and methods of the present invention provide the ability to distinguish between species as shown in FIG. 2B. Thus, it may be seen that the response profile observed from each of the two L. decemlineata EcR/USP heterodimers (FIG. 2B) varies considerably from those seen with the D. melanogaster EcR/USP heterodimers (FIG. 2A). Thus, for Leptinotarsa, RH-2485 evokes a much higher fold-induction (up to 25-fold) from the L. decemlineata heterodimers. By contrast, the response of the LdEcR/LdUSP to murA and 20E may be relatively modest compared to DmEcR/DmUSP, with minimal induction by makA.

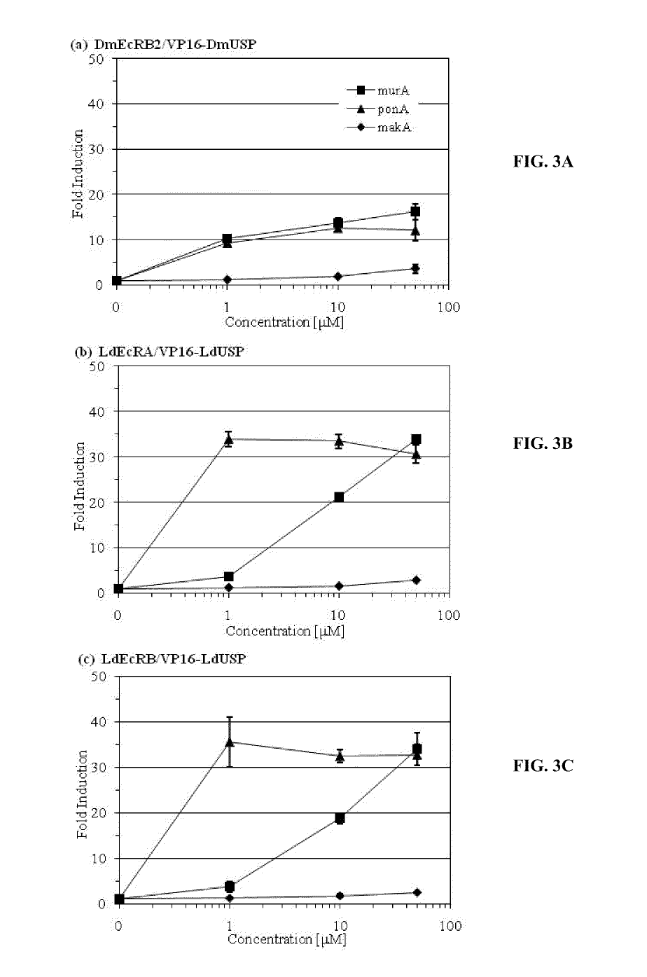

In certain embodiments, the systems and methods of the present invention may be used to measure the potency of natural and nonsteroidal agonists by comparing the dose response of species-specific contstructs. For example, FIG. 3 shows embodiments of the methods and systems of the present invention depicting evaluation of a D. melanogaster EcR-B2/USP complex as compared to two LdEcR/USP complexes. Thus, as shown in FIG. 3, a variety of agonists may be tested. FIG. 3 shows evaluation of three natural ecdysteroids: muristerone A (murA), ponasterone A (ponA), and makisterone A (makA) with receptors from both species. In an embodiment, MurA may be significantly more potent with receptors of D. melanogaster than with those of L. decemlineata. For example, whereas the DmEcR/USP heterodimer may achieve a maximal response in the range of 1-10 μM murA, the LdEcR/USP heterodimer may require about 50 μM murA to attain a maximal response. Nevertheless, the maximal induction evoked by murA at 50 μM with L. decemlineata may still be significant, e.g., over 30-fold. In some cases, receptors from both species were maximally induced by the same amount of an agonist (e.g., 1 μM ponA). Also, in some cases receptors from both species may not respond to an agonist. For example, in an embodiment, neither species responds strongly to mak A, even at 50 μM.

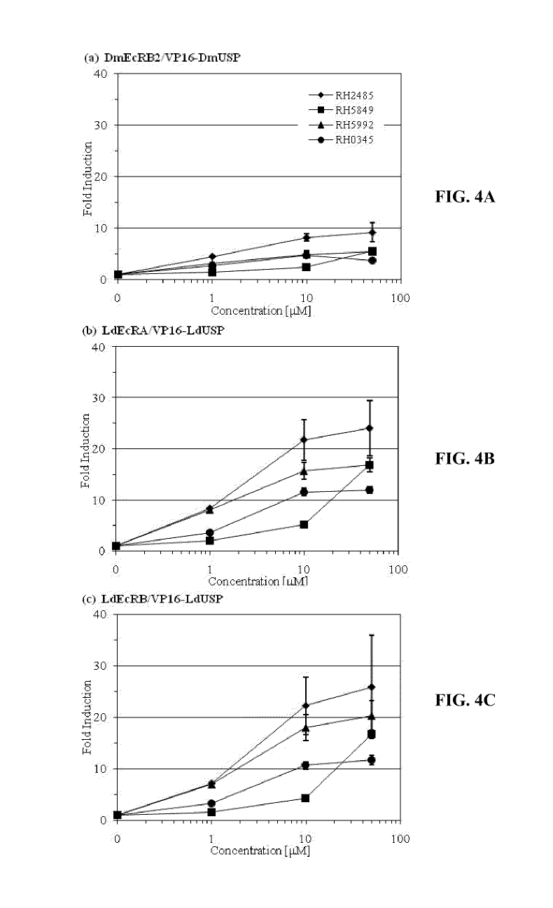

FIG. 4 shows evaluation of four nonsteroidal ecdysteroid agonists, RH-0345, RH-2485, RH-5849 and RH-5992 over a range of dosages with receptors from both species. It can be seen that the methods and systems of the present invention may allow for differentiation of species specific increases in transcription by such non-steroidal agonist.

Thus, as shown in FIG. 4, the maximal fold-induction evoked by nonsteroidal compounds may be considerably higher among the LdEcR dimers than for the compared DmEcR-B2/USP heterodimer. Except for RH5849, each of the RH compounds can evoke a maximal induction at 10 μM with the LdEcR/USP dimers that is greater than 10-fold. In an embodiment, the order of fold-induction obtained for the pooled results (i.e. LdEcR-A and LdEcR-B) is RH2485=RH5992>RH0345>RH5849, one-way ANOVA, P≦0.01). By contrast, the Drosophila receptor may show a more modest induction with all of the nonsteroidal ecdysteroid agonists and, in some embodiments, not exceed 10-fold (FIG. 4A).

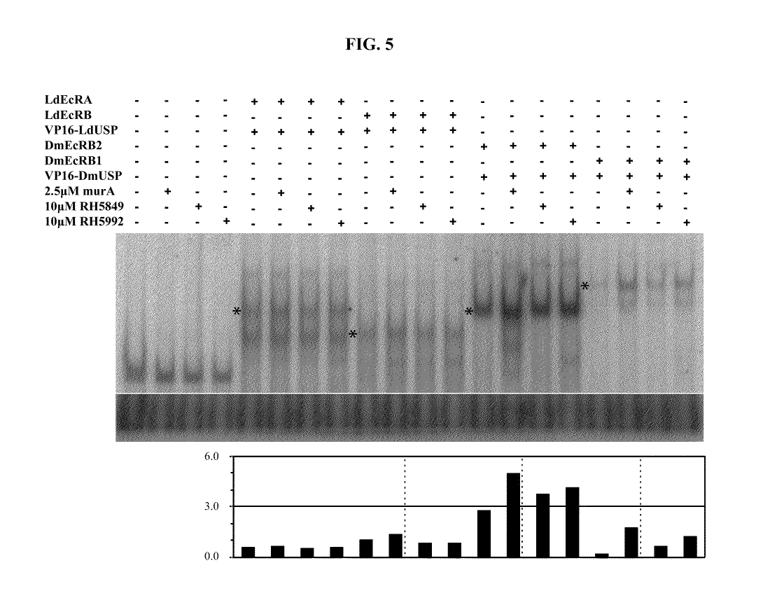

The methods and systems of the present invention may also be used to analyze the interaction of agonists with receptor complexes on the molecular level. For example, as depicted in FIG. 5C, in an embodiment, an electrophoretic mobility shift assay (EMSA) may be performed using cell culture extracts expressing D. melanogaster EcR-B1/DmUSP and EcR-B2/DmUSP or the L. decemlineata EcR/USP combinations to verify their interaction with the hsp27 response element. The observed shifts associated with the hsp27 EcRE may, in certain embodiments, reveal that DmEcR-B1/VP16-DmUSP displays an increased shift intensity in the presence of agonist and that DmEcR-B2/VP16-DmUSP is modestly increased by the presence of agonist (FIG. 5) [9]. In contrast, under identical experimental conditions, the two Leptinotarsa EcR/USP complexes may show little change in shift intensity when an agonist was present.

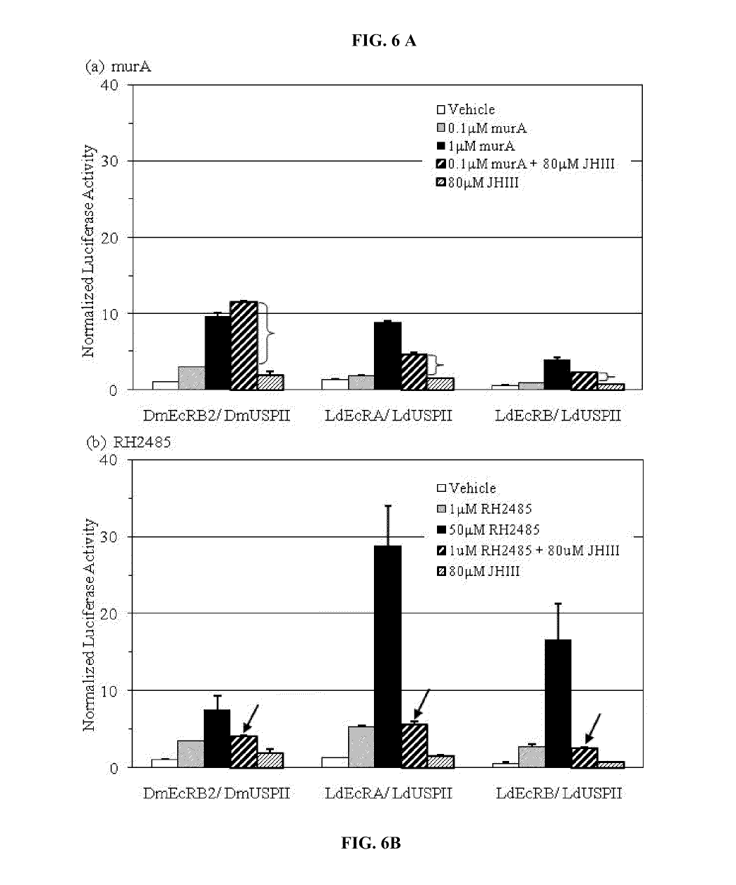

In yet other embodiments, the methods and systems of the present invention may be used to evaluate species-specific potentiation of EcR mediated transcription. For example, FIG. 6 shows an embodiment of the use of the methods and systems of the present invention to evaluate the effect of juvenile hormone on EcR/USP transcriptional activity in two species: Drosophila and Leptinotarsa.

Thus, when Chinese Hamster Ovary (CHO) cells expressing the Drosophila EcR/USP complex are challenged with JH-III alone, no effect on transcriptional activity is observed. However, the simultaneous presence of JH-III in a cell culture medium that already contains ecdysteroids reduces the dosage level of ecdysteroids necessary to achieve maximal transcriptional activity by about tenfold (FIG. 6A). In other words, JH-III potentiates the responsiveness of the EcR/USP complex to ecdysteroids (see e.g., U.S. patent application Ser. No. 10/929,090, incorporated by reference herein in its entirety). Using the methods and systems of the present invention, a submaximal dosage of murA (e.g., 0.1 μM) together with JH-III may be simultaneously tested with cells expressing the DmEcRB2/DmUSPII complex as well as LdEcRA/LdUSP and LdEcRB/LdUSP complexes. In an embodiment, using the Leptinotarsa system, there may be a partial and significant potentiation of submaximal murA by JH-III, although the overall potentiation may be quantitatively different than seen with the Drosophila system; P≧0.01, t-test (FIG. 6A). Interestingly, the nonsteroidal agonist, RH-2485 may not exhibit potentiation by JH-III in either D. melanogaster and L. decemlineata (FIG. 6B). Thus, in at least certain embodiments, potentiation by JH-III may not be a general cellular effect, but may depend upon the specific agonist-EcR interaction.

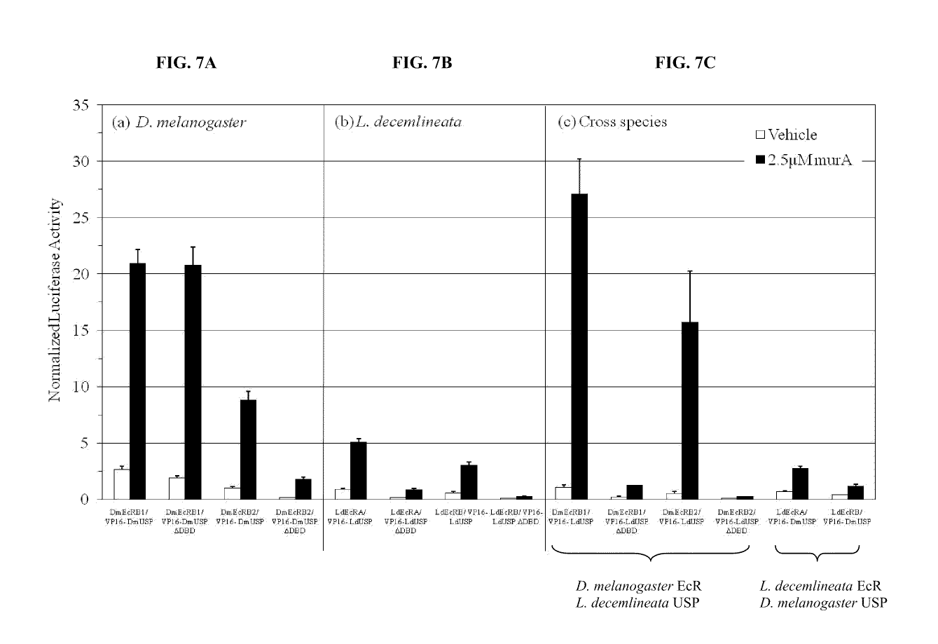

In certain embodiments, the constructs of the present invention may provide either reduced, or significantly enhanced, transcriptional activation and/or potentiation. For example, as illustrated in FIG. 7, when a VP16-DmUSP/ΔDBD is tested with the three D. melanogaster EcR isoforms, the EcR-A and EcR-B2 heterodimers may result in relatively inactive dimers (FIG. 7A). Still in certain embodiments, DmUSP/ΔDBD may retain nearly normal activity when paired with EcR-B1. In an embodiment, when the analogous VP16-LdUSP/ΔDBD is paired with LdEcRA and LdEcRB, the expression of VP16-LdUSP/ΔDBD, may result in a heterodimer with severely reduced transcriptional activity (FIG. 7B). Thus, in certain embodiments, the present invention provides isoform-specific and/or species specific constructs that are relatively insensitive to putative insecticides (e.g., EcRA and/or EcRB paired with VP16-DmUSP/ΔDBD; LdEcRA and LdEcRB paired with VP16-LdUSP/ΔDBD) and/or constructs that display good activation (e.g., EcRB2 paired with VP16-DmUSP/ΔDBD) (FIG. 7A).

In yet other embodiments, cross-species complexes may be used to modify EcR-mediated transcription (e.g., FIG. 7C). As shown in FIG. 7C, the Drosophila EcR-B1 and EcR-B2 isoforms may display a higher level of ligand-dependent (induced) transcriptional activity with VP16-LdUSP than with the equivalent VP16-DmUSP, resulting in a complex with supra-maximal stimulation. In addition, in certain embodiments, the EcR-B1 and EcR-B2 isoforms may display a lower level of ligand-independent (basal) transcriptional activity with VP16-LdUSP than with VP16-DmUSP. In additional and/or alternative embodiments, in contrast to the VP16-DmUSP/ΔDBD, the VP16-LdUSP/ΔDBD may form a relatively inactive dimer with DmEcR-B1. Also, in certain embodiments, VP16-DmUSP may consistently evokes a lower quantitative level of transcriptional activity, with both its own EcR isoforms, and with the two L. decemlineata EcR isoforms.

Thus, embodiments of the present invention allow for the controlled assessment of species specificity to agonist and potentiator induced increases in EcR-mediated transcription. Compounds that are able to modulate EcR-mediated transcription (either as agonists or potentiators) may be insecticidal. The ability to design species-specific insecticides allows for targeting certain insects (e.g., beetles) while leaving other insects (e.g., pollinating bees) unaffected. For example, comparison of the Leptinotarsa and Drosophila EcR/USP heterodimer may reveal a variety of distinctions between them in terms of quantitative level of transcriptional activity, ligand responsiveness, and capability for potentiation by JHIII. These findings are generally consistent with expectations from other in vivo and biochemical work with the two species' receptors and indicate that the CHO cell culture assay system can be employed to characterize individual insect EcR/USP heterodimers for their responsiveness to agonists and potentiators.

In an embodiment, the present invention provides a system (e.g., cell culture) that can be used as a screening assay for novel agonists. Interestingly, the fold-induction evoked by the tested non-steroidal RH compounds on transcriptional activity of L. decemlineata EcR approximately corresponded with their ligand affinity [12, 19], although while RH-0345 is not the most efficacious of the RH compounds in the cell culture assay, it is highly toxic of these compounds in CPB, owing to its relative persistence in target tissues [36]. Thus, other factors, in addition to induction of EcR may be important to the overall toxicity of a compound. In a embodiment, however, ligand potency may be the best primary criterion for isolating insecticidal candidates within a given species even if fold-induction is modest. The potency of RH-0345 with the LdEcR isoforms in the methods and systems of the invention is similar to those of RH-2485 and RH-5992 and all three of these RH compounds showed greater potency and efficacy than RH-5849, which is weakly toxic in CPB. Notably, all of the RH compounds yield a higher fold-induction with the L. decemlineata receptor than with the receptor of D. melanogaster, which is relatively unresponsive to the effects of RH compounds [37], thus suggesting that fold induction can serve as a basis for predicting differences in the toxicity of a compound between species. Interestingly, the weak inductive effects of the natural ecdysteroids (murA, ponA, makA and 20E) further suggest there may be a lack of correspondence between fold-induction and ligand affinity, as the affinities of the natural ecdysteroids for EcR are higher than the affinities of the diacylhydrazines [12].

The differences in fold-induction observed between the natural steroids and the nonsteroidal agonists is expected in part, since these agonist classes involve different amino acid interactions in the ligand-binding pocket. Nevertheless, both D. melanogaster and L. decemlineata EcR carry the same residue at each of the putative binding sites ascribed to the RH compounds [8], consistent with the suggestion that other features of the ligand-binding pocket account for species differences in responsiveness to RH compounds [13].

As shown in detail herein, transcriptional activity levels may vary widely among the three Drosophila EcR isoforms and the two EcR Leptinotarsa isoforms. Such quantitative differences may prove important for in vivo functions. In Manduca, the presence of a B isoform increases transcriptional activity normally mediated by the A isoform alone, heightening the possible relevance of these differences for in vivo regulation [38]. Thus, using the methods and systems of the present invention, it is apparent that Drosophila and Leptinotarsa USP are not interchangeable in terms of transcriptional activity. Interestingly, however, USP does not appear to strongly affect ligand affinity when tested in cross-species dimers [12]. Species-specific differences in USP structure have been implicated in the regulation of developmental events associated with larval growth and subsequent metamorphosis [45]. The effects observed in cross-species EcR/USP dimers further suggests that USP may play a role in determining the quantitative level of transcriptional activity.

The methods and systems of the present invention may be used to screen for compounds that have the ability to potentiate agonist-induced EcR-mediated transcription in a species specific manner. The effects of potentiation may result from a low-affinity interaction between the EcR/USP complex and JH-III. A similar effect for the Drosophila EcR/USP has been observed for methyl farnesoate and other substrates within the mevalonate pathway [14]. Also, the ability of JH-III to potentiate ecdysteroid inducibility has also been observed with polychlorinated biphenyls (PCBs) whose activity is associated with members of the basic helix-loop-helix Per-Arnt-Sim (bHLH-PAS) transcription factor family [46]. Members of this family, in turn, include the Drosophila Methoprene-tolerant (MET) gene product [47] and MET is known to bind to JH-III [48]. Mutations of the MET gene in Drosophila block the normally lethal effects of methoprene application [47]. Mammalian bHLH-PAS transcription factors bind to nuclear receptors, leaving the possibility for a MET/EcR/USP interaction. A physical interaction between MET and both EcR and USP has been reported [49], though its relevance for the functional effects of JH-III remains to be explored. The homologue of MET in Tribolium castaneum mediates JH action, further raising the possibility of a similar role in modulating ecdysteroid receptor action [50]. Nonsteroidal ecdysteroid agonists are known to confer a markedly different shape upon the ligand-binding pocket of EcR than natural ecdysteroids [8] that could prevent interactions with regulatory cofactors such as MET via the LBD. It is important to recognize that USP itself binds to JH and methyl farnesoate under certain experimental conditions [51]. Alternatively, the effect of RH2485 on EcR is to alter the shape of its ligand-binding pocket, thus blocking a potentiation mediated by USP binding to JH-III. Finally, while MET explains some JH-mediated activities in T. castaneum, it does not account for all of them [50], leaving open the possibility that JH acts via multiple modes of action. The inability to see potentiation with nonsteroidal compounds at least demonstrates that the effects of JH-III cannot be attributed to a generalized cellular action upon the transcriptional complex that includes EcR and USP. Rather, the occurrence of potentiation may depend at least in part, upon the specific agonist.

Thus, in the methods and systems of the present invention, a heterologous mammalian cell culture assay may be used to assess the transcriptional activity of the heterodimeric ecdysteroid receptor. In certain embodiments, species representing two major insect orders: the fruitfly, Drosophila melanogaster (Diptera) and the Colorado potato beetle, Leptinotarsa decemlineata (Coleoptera), may be used. Or, other species of insects may be used.

For example, although several nonsteroidal agonists may evoke a strong response with the L. decemlineata heterodimer that was consistent with biochemical and in vivo evidence, the D. melanogaster receptor's response may be comparatively modest. Conversely, the phytoecdysteroid muristerone A may be more potent with the D. melanogaster heterodimer. The additional presence of juvenile hormone III (JHIII) can potentiate the inductive activity of muristerone A in the receptors from both species, but JHIII may be unable to potentiate the inductive activity of the diacylhydrazine, RH-2485, in the receptor of either species. The effects of USP on ecdysteroid-regulated transcriptional activity may also vary between species. For example, when tested with D. melanogaster EcR isoforms, basal activity was lower and ligand-dependent activity was higher with L. decemlineata USP than with D. melanogaster USP. Thus, the methods and systems of the present invention provide for screening of novel agonists and potentiators as species-targeted insecticidal candidates.

Thus, the methods and systems of the present invention further establishes the utility of the heterologous CHO cell culture system for assessing the effects of agonists/antagonists and other modulators on EcR/USP-mediated transcriptional activity. The insect ecdysteroid receptor is a commercially proven target for insecticidal action and the assay provides a conceptual basis for high throughput screening and identifying compounds that perturb receptor function, not only in terms of classic ecdysteroid agonist functions, but also for those that are capable of mimicking or evoking the potentiation effect induced by JHIII on this assay.

### EXAMPLES

### Example 1

### Materials and Methods

A. Cell Culture, EMSA, and Western Immunoblotting Methodology

All aspects of cell culture methodology, ligand application, transfection, reporter gene measurement, Western immunoblotting and electrophoretic mobility shift assays (EMSAs) have been previously reported [9, 21]. Briefly, Chinese hamster ovary (CHO) cells are grown to confluence and transfected (250 ng each) with: (1) a plasmid vector containing the luciferase gene controlled by the canonical hsp27 ecdsyone response element (hsp27 EcRE) and a weak constitutive promoter [33]; (2) a vector containing β-galactosidase gene controlled by a constitutively active promoter; (3) one of the EcR-encoding vectors described below; and (4) one of the USP-encoding vectors described below. After transfection for 6 h, cells were incubated with or without agonists and/or JHIII for 24 h, cells were harvested, and extracts processed for the studies.

Reagents tested included: muristerone A (murA; Alexis Biochemical), ponasterone A, makisterone A (ponA, makA; AG Scientific, San Diego, Calif.), and juvenile hormone-III (JH-III; Sigma Chemical, St. Louis, Mo.). The diacylhydrazine-based agonists that were tested include RH-0345 (halofenozide), RH-2485 (methoxyfenozide), RH-5849 and RH-5992 (tebufenozide), all >95% pure, and were kindly provided by Rohm and Haas Co. (Spring House, Pa.). Western immunoblots of Leptinotarsa and Drosophila EcR were performed with the 9B9 antibody obtained from the Developmental Studies Hybridoma Bank at the University of Iowa.

Band densities were measured using BioRad Quantity One software from the EMSA and Western immunoblot images. The pixel intensity of the band signal was determined for the defined band area and adjusted relative to one of the signals, as designated, to calculate the relative band density.

B. Vector Description and Construction

All D. melanogaster (Dm) EcR and USP expression vectors and the luciferase (and β-galactosidase reporter gene vectors have been described previously [9, 21]. The expression vectors encoding the natural isoforms of the D. melanogaster EcR (DmEcR) are noted as DmEcR-A, Dm EcR-B1, and DmEcR-B2.

The following protocols were used to construct the L. decemlineata (Ld) EcR cell culture vectors encoding its two natural isoforms (LdEcR-A and LdEcR-B). The LdEcR-A open reading frame was isolated by PCR from pBluescriptKS+LdEcRA [31] using the forward primer 5′-TTTTGGATCCACCATGACCACCATACACTCGATC 3′ (SEQ ID NO: 11) and the reverse primer 5′-TTTTTCTAGACTATGTCTTCATGTCGACGTC-3′ (SEQ ID NO: 12). The underlined portion of the primers represents the inserted BamHI and XbaI restriction sites, respectively. The vector pcDNA3.1+ and the LdEcR-A amplicon were digested with the restriction endonucleases BamHI and XbaI. The digestion products were purified from an agarose gel excision then ligated to create the vector pcDNA3.1+LdEcRA. The LdEcR-B fragment was removed from pBluescriptKS+LdEcR-B [31] and the vector pcDNA3.1 (InVitrogen; Carlsbad, Calif.) was linearized by restriction digest with XbaI and BamHI. Both restriction products were excised from an agarose gel-purified, and then ligated to produce the vector pcDNA3.1-LdEcR-B.

The vectors encoding the D. melanogaster USP (DmUSP) have also been described previously [9]. For these vectors, the N-terminal (A/B) domain of DmUSP was replaced with the VP16 activation domain, since the DmUSP A/B domain displays minimal transcriptional activity in CHO cells [18]. Two constructs were produced: VP16-DmUSP includes the USP DNA-binding domain (DBD), whereas VP16-DmUSP/ΔDBD has had the DBD deleted.

The analogous VP16-LdUSP and VP16-LdUSP/ΔDBD vectors were constructed for this study as follows. The LdUSP and LdUSP/ΔDBD fragments were isolated by PCR from pBluescriptKS+LdUSP [31] using the forward primer 5′-TTTTGAATTCTGCTCGATTTGCGGGGACAAG-3′ (SEQ ID NO: 13) for LdUSP (which is the 5′ end of the DBD-encoding DNA sequence) or 5′-TTTTGAATTCAAGCGGGAGGCGGTTCAAGAA-3′ (SEQ ID NO: 14) (which lies just to the 3′ side of the DBD-encoding sequence). Each primer was paired with the reverse primer 5′-TTTTAAGCTTCTAAGTATCCGACTGGTTTTC-3′ (SEQ ID NO: 15) which is the complement of the 3′ end of the LdUSP LBD. The respective EcoRI and HindIII restriction sites inserted by the PCR primers are underlined. The resulting LdUSP amplicon includes the entire DBD, whereas LdUSP/ΔDBD includes the entire open reading frame beginning at the first amino acid following the LdUSP DBD. Both amplicons and the pVP16 vector were digested with EcoRI and HindIII restriction endonucleases. Ligation of the products into the linearized pVP16 vector (Clontech; Mountain View, Calif.) resulted in the pVP16 LdUSP and pVP16LdUSP/ΔDBD constructs. All constructs were subsequently verified by DNA sequencing.

### Example 2

### Sequence Comparison

The DBDs of the Leptinotarsa and Drosophila EcR and USP receptors are identical at every amino acid position that is conserved among all EcR and USP DBD sequences respectively, and share an overall identity of over 90% in both cases [31]. Therefore, it was expected that the canonical hsp27 EcRE, would allow direct comparisons of agonist inducibility when tested with EcR/USP from each of the two species.

As reported elsewhere, and as shown in FIG. 1, sequence conservation is not as extensively shared in the LBD, where the identity between Drosophila and Leptinotarsa EcR is about 67% [21]. USP LBD conservation is less than 39% between the two species [21]. This divergence in the LBDs of EcR and USP between the two species provided a basis for predicting that their ligand-dependent transcriptional capabilities (AF2) are also different.

The N-terminal (A/B) domains of EcR are also divergent in the two insect species [31] though all of the isoforms from both species share near identity over a stretch of 35-37 amino acids that lie just to the N-terminal side of the DBD (FIG. 1C). The EcR-A isoforms from the two species share a few similar motifs in the middle region of the A/B domain (FIG. 1A) whereas the LdEcR-B shares some identity with DmEcRB1 only in the most N-terminal region (FIG. 1B). DmEcR-B2 is unique except in the aforementioned common region shared by all the isoforms (FIGS. 1B and 1C). The divergence of sequences in the EcR A/B domain, where ligand-independent transcriptional activity (AF1) resides, may indicate why transcriptional activity levels are variable.

### Example 3

### Efficacy of Agonists on Dm and Ld EcR/USP Transcriptional Activity

The basal and ligand-induced properties of the three D. melanogaster isoforms (DmEcR-A, DmEcR-B1 and DmEcR-B2) with the VP16-DmUSP heterodimer were compared with those of the L. decemlineata isoforms (EcR-A and EcR-B) paired with the equivalent VP16-LdUSP construct. In this system, the VP16 activation domain replaces the highly variable N-terminal domain (A/B) of USP from both species, allowing for direct cross-species comparison of activity [18]. Activity was determined by measuring reporter gene (luciferase) activity mediated by the hsp27 EcRE after normalization for cell mass using β-galactosidase activity registered via a constitutive promoter.

In order to compare the efficacy of agonists, maximally-inducing dosages of several ecdysteroids and the most inductive nonsteroidal agonist, RH2485, based on preliminary experiments, were tested. The pattern of response was similar for each of the three D. melanogaster isoforms (FIG. 2A). In all cases, murA (2.5 μM) evoked the strongest fold-induction, and the greatest absolute level of transcriptional activity. RH-2485 also evoked a response from all three Drosophila EcR isoforms, with lesser responses from the natural molting hormone, 20E, and makisterone A, the latter being the most abundant ecdysteroid in late third instar whole body titers of D. melanogaster [34]. The relatively modest response to natural ecdysteroids such as 20E has been noted in previous cell culture studies. Also, differences in the quantitative levels of transcription were previously reported, with Drosophila EcR-B1 showing the highest levels of basal and induced activity, and EcR-A displaying the lowest levels of activity [9].

The response profile observed from each of the two L. decemlineata EcR/USP heterodimers varied considerably from those seen with the D. melanogaster EcR/USP heterodimers (FIG. 2B). RH-2485 evoked a much higher fold-induction (up to 25-fold) from the L. decemlineata heterodimers. By contrast, the response of the LdEcR/LdUSP to murA and 20E was relatively modest compared to DmEcR/DmUSP. A minimal induction was registered by makA.

Differences in normalized induction in this experiment and others are not attributable to differences in cell growth caused by the effects of the individual ligands. The β-galactosidase reporter gene measurements used to normalize transcriptional activity (by providing an estimate of cell mass) varied by less than 20% for all the ligand regimens applied. Also, the absolute β-galactosidase values varied by less than 20% between experiments, indicating that cell growth rates were relatively constant (data not shown).

Immunoblots were also performed with cell extracts expressing the EcR isoforms employed in this study to observe whether transcriptional activity levels are related to expression levels. While the signal evoked from individual isoforms varied to some degree, as noted in previous work [9], the strength of signal did not correlate with differences in transcriptional activity (FIG. 2C). For instance, Drosophila EcR-B1 activity is much higher than EcR-B2 activity, though the EcR-B2 signal is relatively strong on immunoblots. In other words, differences in activity level appear to reflect intrinsic functional differences among the receptor isoforms and species.

In summary, each of the isoforms within a species generated a similar responsiveness to maximal dosages of individual agonists. Whereas the EcR N-terminal domain influences the quantitative level of transcription for a given isoform, it had no effect on relative ligand responsiveness. Importantly, the relative induction to individual agonists was species-specific for all of the tested ligands, and the responsiveness to RH-2485 was much higher in Leptinotarsa than in Drosophila, whereas Drosophila EcR/USP was more responsive to murA than any other agonist.

### Example 4

### Potency of Selected Ecdysteroids and Nonsteroidal Ecdysteroid Agonists on Transcriptional Activity in Two Species

The potency of natural and nonsteroidal agonists was further evaluated by comparing the dose response of the D. melanogaster EcR-B2/USP complex with the two LdEcR/USP complexes.

Three natural ecdysteroids, muristerone A (murA), ponasterone A (ponA), and makisterone A (makA), were tested in receptors from both species (FIG. 3A-3C). It can be seen that MurA was significantly more potent with receptors of D. melanogaster than with those of L. decemlineata. Whereas the DmEcR/USP heterodimer achieves a maximal response in the range of 1-10 μM murA, the LdEcR/USP heterodimer requires about 50 μM murA to attain a maximal response. Nevertheless, the maximal induction evoked by murA at 50 μM was over 30-fold with L. decemlineata. Receptors from both species were maximally induced by 1 μM ponA, and neither species responded strongly to mak A, even at 50 μM.

Four nonsteroidal ecdysteroid agonists, RH-0345, RH-2485, RH-5849 and RH-5992, also were tested over a range of dosages with receptors from both species (FIGS. 4A-4C). The maximal fold-induction evoked by nonsteroidal compounds was considerably higher among the LdEcR dimers than it was for the compared DmEcR-B2/USP heterodimer. Except for RH5849, each of the RH compounds evoked a maximal induction at 10 μM with the LdEcR/USP dimers that was greater than 10-fold. The order of fold-induction obtained for the pooled results (i.e. LdEcR-A and LdEcR-B) was RH2485=RH5992>RH0345>RH5849, one-way ANOVA, P≦0.01). By contrast, the Drosophila receptor showed a more modest induction with all of the nonsteroidal ecdysteroid agonists and never exceeded 10-fold (FIG. 4A).

An electrophoretic mobility shift assay (EMSA) was also performed using cell culture extracts expressing D. melanogaster EcR-B1/DmUSP and EcR-B2/DmUSP or the L. decemlineata EcR/USP combinations to verify their interaction with the hsp27 response element. The observed shifts associated with the hsp27 EcRE revealed that DmEcR-B1/VP16-DmUSP showed an increased shift intensity in the presence of agonist and that DmEcR-B2/VP16-DmUSP was modestly increased by the presence of agonist (FIG. 5) [9]. Under identical experimental conditions, the two Leptinotarsa EcR/USP complexes showed little change in shift intensity when an agonist was present. The variability among the individual EcR/USP pairings could be attributed to the selected conditions, which had been optimized for testing Drosophila EcR/USP.

### Example 5

### Species-Specific Potentiation of Juvenile Hormone on EcR/USP Transcriptional Activity

When CHO cells expressing the Drosophila EcR/USP complex are challenged with JH-III alone, no effect on transcriptional activity is observed [9]. However, the simultaneous presence of JH-III in a cell culture medium that already contains ecdysteroids reduces the dosage level of ecdysteroids necessary to achieve maximal transcriptional activity by about tenfold. In other words, JH-III potentiates the responsiveness of the EcR/USP complex to ecdysteroids [9, 14, 21]. Using the same paradigm employed for measuring potentiation in the Drosophila system, a submaximal dosage of murA (0.1 μM) together with JH-III was simultaneously tested with cells expressing the LdEcR/USP heterodimer. Under these conditions, a partial and significant potentiation by JH-III was observed in the L. decemlineata receptor; P≧0.01, t-test (FIG. 6A).

The potentiation testing paradigm was then modified by testing the nonsteroidal agonist, RH-2485 instead of murA. Interestingly, no potentiation by JH-III was seen in either D. melanogaster and L. decemlineata, using RH-2485 as an agonist (FIG. 6B). This result indicates that potentiation may not be a general effect, but may depend upon the species-specific agonist-EcR interaction.

### Example 6

### Effect of L. decemlineata and D. melanogaster USP Constructs on Ecdysteroid-Inducible Transcriptional Activity

As noted, when VP16-DmUSP/ΔDBD is tested with the three D. melanogaster EcR isoforms, EcR-A and EcR-B2 heterodimers form a relatively inactive dimer [9] (FIG. 7A). However, DmUSP/ΔDBD retains nearly normal activity when paired with EcR-B1, indicating that the nature of the EcR/USP interaction is isoform-specific [9, 35] (FIG. 7A). The analogous VP16-LdUSP/ΔDBD was tested with LdEcRA and LdEcRB. In both cases, the expression of VP16-LdUSP/ΔDBD, as verified by immunoblots (data not shown), resulted in a heterodimer with severely reduced transcriptional activity (FIG. 7B).

In order to compare the capabilities of DmUSP and LdUSP further, cross-species heterodimers were tested for transcriptional activity (FIG. 7C). At least four functional differences were observed: (1) the Drosophila EcR-B1 and EcR-B2 isoforms display a higher level of ligand-dependent (induced) transcriptional activity with VP16-LdUSP than with the equivalent VP16-DmUSP; (2) the same EcR-B1 and EcR-B2 isoforms display a lower level of ligand-independent (basal) transcriptional activity with VP16-LdUSP than with VP16-DmUSP; (3) VP16-LdUSP/ΔDBD forms a relatively inactive dimer with DmEcR-B1, unlike VP16-DmUSPΔDBD; and (4) the VP16-DmUSP consistently evokes a lower quantitative level of transcriptional activity, with both its own EcR isoforms, and with the two L. decemlineata EcR isoforms.

It will be understood that each of the elements described above, or two or more together, may also find utility in applications differing from the types described. While the invention has been illustrated and described as nucleic acid constructs for the identification of species specific insecticides or other modulators of insect growth and/or development, it is not intended to be limited to the details shown, since various modifications and substitutions can be made without departing in any way from the spirit of the present invention. As such, further modifications and equivalents of the invention herein disclosed may occur to persons skilled in the art using no more than routine experimentation, and all such modifications and equivalents are believed to be within the spirit and scope of the invention as described herein. All patents and published patent applications referred to in this document are incorporated by reference in their entireties herein.

### REFERENCES

1. Thummel, C S (2002) Ecdysone-regulated puff genes 2000. Insect Biochem. Mol. Biol. 32, 113-120.
2. M. Lezzi, T. Bergman, J.-F. Mouillet, and V. C. Henrich (1998) The ecdysone receptor puzzle. Arc Insect Biochem. Physiol. 41, 99-106.
3. iddiford, L M, P Cherbas, and J W Truman (2000) Ecdysone receptors and their biological actions. Vitam Horm. 60:1-73.
4. Billas, I, and D Moras (2005) Ligand-binding pocket of the ecdysone receptor. Vitam. Horm. 73:101-29.
5. Henrich, V (2005) “The Ecdysteroid Receptor”. In: Comprehensive Insect Physiology, Biochemistry, and Molecular Biology Series, Volume 3. Elsevier Press, Oxford, UK. pp. 243-286.
6. Palli, S R, R E Hormann, U Schlattner, and M Lezzi (2005) Ecdysteroid receptors and their applications in agriculture and medicine. Vit. and Hormones 73, 60-100.
7. Mouillet. J-F, V. C. Henrich, M. Lezzi, and M. Vogtli (2001) Differential control of gene activity by isoforms A, B1, and B2 of the Drosophila ecdysone receptor. Eur. J. Biochem., 268: 1811-1819.
8. Billas I M, Iwema T, Garnier J M, Mitschler A, Rochel N, Moras D. 2003. Structural adaptability in the ligand-binding pocket of the ecdysone hormone receptor. Nature 426:91-6.
9. Beatty, J, T Fauth, J L Callender, M Spindler-Barth, and V C Henrich (2006) Analysis of transcriptional activity mediated by the Drosophila melanogaster ecdysone receptor isoforms in a heterologous cell culture system. Insect Mol. Biol., 15, 785-795.
10. Graham L D, Pilling P A, Eaton R E, Gorman J J, Braybrook C, Hannan G N, Pawlak-Skrzecz A, Noyce L, Lovrecz G O, Lu L, Hill R J (2007a). Purification and characterization of recombinant ligand-binding domains from the ecdysone receptors of four pest insects. Protein Expr Purif. 53:309-24.
11. Graham, L D, W M Johnson, A Pawlak-Skrzeca, RE Eaton, M Bliese, L Howell, G N Hannan, and R J Hill (2007b) Ligand binding by recombinant domains from insect ecdysone receptors. Insect Biochem Mol. Biol. 37, 611-626.
12. Minakuchi, C, T. Ogura, H. Miyagawa, and Y. Nakagawa (2007) Effects of the structures of ecdysone receptor (EcR) and ultraspiracle (USP) on the ligand-binding activity of the EcR/USP heterodimer. J. Pesticide Sci. 32, 379-384.
13. Carmichael, J A, M C Lawrence, L D Graham, P A Pilling, V C Epa, L Noyce, G Lovrecz, D A Windler, A Pawlak-Skrezecz, R E Eaton, G N Hannan, and RJ Hill (2005) The X-ray structure of a hemipteran ecdysone receptor ligand-binding domain: comparison with a lepidopteran ecdysone receptor ligand-binding domain and implications for insecticide design. J. Biol. Chem. 280, 22258-22269.
14. V C Henrich, J M Beatty, H Ruff, J Callender, M Grebe, and M Spindler-Barth (2009) The multidimensional partnership of EcR and USP. In: Ecdysones, Structures and Functions. Springer-Verlag, in press.
15. Grebe, M, S. Przibilla, V. C. Henrich, and M. Spindler-Barth (2003) Characterization of the ligand-binding domain of the ecdysteroid receptor from Drosophila melanogaster. Biol.Chem. 384, 93-104.
16. Nakagawa, Y (2005) Nonsteroidal ecdysone agonists. Vitam. Horm. 73, 131-173.
17. Christopherson K S, Mark M R, Bajaj V, Godowski P J. 1992. Ecdysteroid-dependent regulation of genes in mammalian cells by a Drosophila ecdysone receptor and chimeric transactivators. Proc Natl Acad Sci USA. 1992; 89:6314-8.
18. Yao, T. P, B. M. Forman, Z. Y. Jiang, L. Cherbas, J. D. Chen, M. McKeown, P. Cherbas, and R. M. Evans (1993) Functional ecdysone receptor is the product of EcR and ultraspiracle genes. Nature 366: 476-479.
19. Hormann, R. E., Smagghe, G. & Nakagawa, Y. (2008). Multidimensional quantitative structure-activity relationships of diacylhydrazine toxicity in Spodoptera exigua, Chilo suppressalis, and Leptinotarsa decemlinata, QSAR and Combinatorial Sciences, in press.
20. Wheelock, C. E., Nakagawa, Y., Harada, T., Oikawa, N., Akamatsu, M., Smagghe, G., Stefanou, D., Iatrou, K. & Swevers, L. (2006). High throughput screening of ecdysone agonists using a reporter gene assay followed by 3-D QSAR analysis of the molting hormonal activity. Bioorganic and Medical Chemistry, 14, 1143-1159.)
21. Henrich, V. C., E. Burns, D. P. Yelverton, E. Christensen, and C. Weinberger (2003) Juvenile hormone potentiates ecdysone receptor-dependent transcription in a mammalian cell culture system, Insect Biochem. Mol. Biol., 33, 1239-1247.
22. Dinan, L, T Savchenko, and P Whiting (2001) On the distribution of phytoecdysteroids in plants, Cell Mol. Life Sci. 58, 1121-1132.
23. Elbrecht, A, Y Chen, T Jurgens, O D Hensens, D L Zink, H T Beck, M J Balick, and R Borris (1996), 8-O-acetylharpagide is a nonsteroidal ecdysteroid agonist. Insect Biochem. Mol. Biol. 26, 519-523.
24. Staal, G B (1986) Anti juvenile hormone agents. Ann. Rev. of Entomol. 31, 391-429.
25. Smagghe, G. & Degheele, D. (1994a). Action of a novel nonsteroidal ecdysteroid mimic, tebufenozide (RH-5992), on insects of different orders. Pesticide Science, 42, 85-92.
26. Smagghe, G. & Degheele, D. (1994b). The significance of pharmacokinetics and metabolism to the biological activity of RH-5992 (tebufenozide) in Spodoptera exempta, Spodoptera exigua, and Leptinotarsa decemlineata. Pesticide Biochemistry and Physiology, 49, 224-234.
27. Nakagawa, Y., Smagghe, G., Tirry, L., Kugimiya S., Ueno, T. and Fujita, T. (1999). Quantitative structure-activity studies of insect growth regulators: XVI. Substituent effects of dibenzoylhydrazines on the insecticidal activity to Colorado potato beetle Leptinotarsa decemlineata. Pesticide Science, 55, 909-918).
28. Alyokhin A, Dively G, Patterson M, Castaldo C, Rogers D, Mahoney M, Wollam, J. (2007), Resistance and cross-resistance to imidacloprid and thiamethoxam in the Colorado potato beetle Leptinotarsa decemlineata. Pest Management Sci. 63: 32-41.
29. Khelifi, M., Lague, C., de Ladurantaye, Y. (2007). Physical control of colorado potato beetle: A review, Applied Engineering in Agric. 23: 557-569.
30. Laudet, V and F Bonneton (2005) Evolution of nuclear hormone receptors in insects. In: Comprehensive Insect Physiology, Biochemistry, and Molecular Biology Series, Volume 3, Elsevier Press, Oxford, UK. pp. 287-318.
31. Ogura T, Minakuchi C, Nakagawa Y, Smagghe G, Miyagawa H. (2005) Molecular cloning, expression analysis and functional confirmation of ecdysone receptor and ultraspiracle from the Colorado potato beetle Leptinotarsa decemlineata. FEBS J. 272:4114-28.
32. Iwema, T, IML Billas, Y Beck, F Bonneton, H Nierengarten, A Chaumot, G Richards, V Laudet, and D Moras (2007) Structural and functional characterization of a novel type of ligand-independent RXR-USP receptor, EMBO J. 20, 3770-3782.
33. G Riddihough and Pelham, HRB (1987) An ecdysone response element in the Drosophila hsp27 promoter, EMBO J 6: 3729-3734.
34. Pak M D and Gilbert L I (1984) A developmental analysis of ecdysteroids during the metamorphosis of Drosophila melanogaster. J. Liq Chromatogr 20, 2591-2597.
35. Ghbeish N, Tsai C C, Schubiger M, Zhou J Y, Evans R M, McKeown M. (2001) The dual role of ultraspiracle, the Drosophila retinoid X receptor, in the ecdysone response, Proc. Natl. Acad. USA, 98:3867-3872.
36. Nakagawa, Y., Smagghe, G., van Paemel, M., Tirry, L., Fujita, T. (2001). Quantitative structure-activity studies of insect growth regulators: XVIII. Effects of substituent on the aromatic moiety of dibenzoylhydrazines on larvicidal activity against the Colorado potato beetle Leptinotarsa decemlineata. Pest Management Science 57: 858-865.
37. Nakagawa, Y., C Minakuchi, K Takahashi, and T Ueno (2002) Inhibition of [3H] ponasterone A binding by ecdysone agonists in the intact Kc cell line. Insect Biochem. Mol. Biol. 32, 175-180.
38. Hiruma, K, and LM Riddiford (2004) Differential control of MHR3 promoter activity by isoforms of the ecdysone receptor and inhibitory effects of E75A and MHR3. Dev Biol. 272:510-21.
39. Azotei, A, and M Spindler-Barth (2009) DNA affects Ligand Binding of the cdysone Receptor of Drosophila melanogaster. Mol. Cell. Endocrinol, in press.
40. Ruff, R, C Tremmel, and M Spindler-Barth (2009) Transcriptional activity of ecdysone receptor isoforms is regulated by modulation of receptor stability and interaction with AB and C domains of the heterodimerization partner Ultraspiracle, Arch. Insect Biochem. Physiol, in press.
41. Braun, S, A Azotei, and M Spindler-Barth (2009) DNA Binding Properties of Drosophila Ecdysone Receptor Isoforms and its Modification by the Heterodimerization Partner Ultraspiracle. Arch. Insect Biochem. Physiol., in press.
42. Nieva C, Gwozdz T, Dutko-Gwozdz J, Wiedenmann J, Spindler-Barth M, Wieczorek E, Dobrucki J, Dus D, Henrich V, Ozyhar A, Spindler K D. (2005) Ultraspiracle promotes the nuclear localization of ecdysteroid receptor in mammalian cells. Biol. Chem. 386:463-470.
43. Reinking, J, M M S Lam, K Pardee, H M Sampson, S Liu, P Yang, S Williams, W White, G Lajoie, A Edwards, and H M Krause (2005) The Drosophila nuclear receptor E75 contains heme and is gas responsive. Cell 122, 195-207.
44. Koelle, M R, W S Talbot, W A Segraves, M T Bender, P Cherbas, and D S Hogness (1991) The Drosophila EcR gene encodes an ecdysone receptor, a new member of the steroid receptor superfamily. Cell 4, 59-77.
45. Henrich, V. C., M. E. Vogtli, C. Antoniewski, M. Spindler-Barth, S. Przibilla, M. Noureddine, and M. Lezzi (2000) Developmental effects of chimeric ultraspiracle gene derived from Drosophila and Chironomus. Genesis, 28:125-133.
46. Oberdorster, E, D M Cottam, F A Wilmot, M J Milner, and J A McLachlan (1999) Interaction of PAHs and PCBs with ecdysone-dependent gene expression and cell proliferation. Toxicol. AppL Pharmacol. 160: 101-108.
47. Ashok, M, C Turner, and T G Wilson (1998) Insect juvenile hormone resistance gene homology with the bHLH-PAS family of transcriptional regulators. Proc. Natl. Acad. Sci. USA 95: 2761-2766.
48. Miura K, M Oda, S Makita, and Y Chinzei (2005) Characterization of the Drosophila Methoprene-tolerant gene product. Juvenile hormone binding and ligand-dependent gene regulation, FEBS J 272, 1169-1178.
49. Li, Y, Z Zhang, G E Robinson, and S R Palli (2007) Identification and characterization of a juvenile hormone response element and its binding proteins. J. Biol. Chem. 282: 37605-37617.
50. Konopova, B, and M Jindra (2007) Juvenile hormone resistance gene Methoprene-tolerant controls entry into metamorphosis in the beetle Tribolium castaneum. Proc. Nati. Acad. Sci USA 104:10488-10493.
51. Jones G, Jones D, Teal P, Sapa A, Wozniak M. 2006. The retinoid-X receptor ortholog, ultraspiracle, binds with nanomolar affinity to an endogenous morphogenetic ligand, FEBS J., 273:4983-4989.

That which is claimed is:

1. A system for the identification of a compound that can modulate insect growth and/or development in a species-specific and/or stage specific manner comprising:
an isolated nucleic acid molecule that encodes a polypeptide comprising an ecdysone receptor (EcR) isoform from a distinct species; and
a second isolated nucleic acid molecule comprising a DNA sequence that encodes a polypeptide comprising at least a portion of an Ultraspiracle (USP) protein from a distinct species.

2. The system of claim 1, comprising a cell transfected with the DNA sequence that encodes a polypeptide comprising an ecdysone receptor (EcR) isoform from a first species and nucleic acid molecule comprising a DNA sequence that encodes a polypeptide comprising at least a portion of an Ultraspiracle (USP) protein from a second species.

3. The system of claim 1, comprising a cell transfected with the DNA sequence that encodes a polypeptide comprising an ecdysone receptor (EcR) isoform from a first species and nucleic acid molecule comprising a DNA sequence that encodes a polypeptide comprising at least a portion of an Ultraspiracle (USP) protein from the first species and another cell transfected with the DNA sequence that encodes a polypeptide comprising an ecdysone receptor (EcR) isoform from a second species and nucleic acid molecule comprising a DNA sequence that encodes a polypeptide comprising at least a portion of an Ultraspiracle (USP) protein from the second species.

4. The system of claim 1, wherein the species comprise at least one of Drosophila melanogaster, Leptinotarsa decemlineata, Chroristoneura fumerifana, Manduca sexta, Locusta migratoria, Heliothis virescens, Apis mellifera, Aedes aegypti, or Tenebrio molitor.

5. The system of claim 1, wherein the nucleic acid sequence that encodes the USP polypeptide comprises a sequence that encodes:
(a) a mammalian nuclear receptor transactivation domain; and
(b) a hinge region and a ligand binding domain of an insect USP protein.

6. The system of claim 5, wherein the nucleic acid sequence that encodes the USP polypeptide further comprises a sequence that encodes the DNA binding domain of the USP protein.

7. The system of claim 1, further comprising an isolated nucleic acid sequence comprising a reporter gene operably linked to a hormone response element.

8. The system of claim 1, further comprising a mammalian cell transfected with the isolated nucleic acid molecules of claim 1.

9. The system of claim 1, wherein at least one of the isolated nucleic acid molecules is a Drosophila melanogaster EcR isoform comprises at least one of EcRA, EcRB1, or EcRB2.

10. The system of claim 1, wherein at least one of the isolated nucleic acid molecules is a Leptinotarsa decemlineata EcR isoform comprises at least one of EcRA or EcRB.

11. The system of claim 1, wherein at least one of the isolated nucleic acid molecules is a VP16-Leptinotarsa decemlineata USP (VP16-LdUSP).

12. The system of claim 1, wherein at least one of the isolated nucleic acid molecules is a VP16-Drosophila melanogaster USP (VP16-DmUSP).

13. The system of claim 1, further comprising an EcR agonist.

14. The system of claim 13, wherein the agonist comprises muristerone A, Ponasterone A, makisterone A, RH2485, RH5849, RH5992, or RH0345.

15. The system of claim 1, further comprising a compound to be tested for its ability to potentiate EcR-mediated transcription by an ecdysteroid agonist.

16. A method for the identification of a compound that can modulate insect growth and/or development in a species-specific and/or stage specific manner comprising:
(a) transfecting a first mammalian cell population with a first vector comprising a nucleotide sequence that encodes at least a portion of a USP polypeptide, and a second vector comprising a DNA sequence that encodes a polypeptide comprising an ecdysone receptor (EcR) isoform;
(b) transfecting a second mammalian cell population with a vector comprising a nucleotide sequence that encodes at least a portion of a USP polypeptide and a second vector comprising a DNA sequence that encodes for a polypeptide comprising an ecdysone receptor (EcR) isoform; and
(c) comparing the ability of a compound of interest to increase ecdysteroid-mediated transcription of the reporter gene in the first and second cell populations.

17. The method of claim 16, wherein the EcR isoform and the USP of step (a) are each from a first species and the EcR isoform and the USP of step (b) are each from a second species.

18. The method of claim 16, wherein at least one of the EcR isoform or the USP of step (a) are from a first species and at least one of the EcR isoform or the USP of step (b) are each from a second species.

19. The method of claim 16, wherein the USP of step (a) and step (b) are each from the same species.

20. The method of claim 16, wherein the USP of step (a) and step (b) are each from different species.

21. The method of claim 16, wherein the EcR of step (a) and step (b) are from the same species.

22. The method of claim 16, wherein the EcR of step (a) and step (b) are from different species.

23. The method of claim 16, wherein the compound of interest is an EcR agonist.

24. The method of claim 23, wherein the agonist comprises muristerone A, Ponasterone A, makisterone A, RH2485, RH5849, RH5992, or RH0345.

25. The method of claim 16, further comprising adding a compound to be tested for its ability to potentiate EcR-mediated transcription by an EcR agonist and measuring whether there is an increase in EcR-mediated transcription when the compound to be tested is added with the EcR agonist, as compared to when only the agonist is added, or when only the compound to be tested for its ability to potentiate EcR-mediated transcription is added, such that a compound that increases ecdysteroid-activated EcR-mediated transcription to a maximum level at agonist concentrations that are normally sub-maximal, but does not directly increase EcR-mediated transcription, is a potentiator compound.

26. The method of claim 16, wherein at least one of the isolated nucleic acid molecules is a Drosophila melanogaster EcR isoform comprises at least one of EcRA, EcRB1, or EcRB2.

27. The method of claim 16, wherein at least one of the isolated nucleic acid molecules is a Leptinotarsa decemlineata EcR isoform comprises at least one of EcRA or EcRB.

28. The method of claim 16, wherein at least one of the isolated nucleic acid molecules is a VP16-Leptinotarsa decemlineata USP (VP16-LdUSP).

29. The method of claim 16, wherein at least one of the isolated nucleic acid molecules is a VP16-Drosophila melanogaster USP (VP16-DmUSP).


SEQUENCE LISTING (15 sequences)

SEQ ID NO: 1 - protein, 197 aa, Drosophilia melanogaster
MLTTSGQQQSKQKLSTLPSHILLQQQLAASAGPSSSVSLSPSSSAALTLHVASANGGARETTSAAAVKDKLRPTPTAIKIEPMPDVISVGTVAGGSSVATVVAPAATTTSNKPNSTAAPSTSAAAANGHLVLVPNKRPRLDVTEDWMSTPSPGSVPSSAPPLSPSPGSQNHSYNMSNGYASPMSAGSYDPYSPTGKT

SEQ ID NO: 2 - protein, 163 aa, Leptinotarsa decemlineata
MTTIHSITSHLGSMDIKHEMIYRDDDVLLVKSEPQLFTSNNGSITVSNLLSTSNFTTNTNNILVATTSNISSSNGLLLANNGLGNAGPFSGFNNSVVSVSQATKRQMTEDFMSSPSPGAISVSAPPLTPSPGPPSQPYTVISNGYSSPMSSGSYDPYSPNGKL

SEQ ID NO: 3 - protein, 226 aa, Drosophilia melanogaster
MKRRWSNNGGFMRLPEESSSEVTSSSNGLVLPSGVNMSPSSLDSHDYCDQDLWLCGNESGSFGGSNGHGLSQQQQSVITLAMHGCSSTLPAQTTIIPINGNANGNGGSTNGQYVPGATNLGALANGMLNGGFNGMQQQIQNGHGLINSTTPSTPTTPLHLQQNLGGAGGGGIGGMGILHHANGTPNGLIGVVGGGGGVGLGVGGGGVGGLGMQHTPRSDSVNSISS

SEQ ID NO: 4 - protein, 86 aa, Leptinotarsa decemlineata
MKRRWNGFRDAAEESSSEVTSSSTLVMSPANSLASADIGDVDLEFWDLDLNHQHHARNLIIQNGYTLTGTIISHTLAKSDTSSMSS

SEQ ID NO: 5 - protein, 17 aa, Drosophilia melanogaster
MDTCGLVAELAHYIDAY

SEQ ID NO: 6 - protein, 37 aa, Drosophilia melanogaster
GRDDLSPSSSLNGYSANESCDAKKSKKGPAPRVQEEL

SEQ ID NO: 7 - protein, 35 aa, Leptinotarsa decemlineata
GREDLSPPSSLNGFSADSCDAKKKKGPTPRQQEEL

SEQ ID NO: 8 - protein, 37 aa, Drosophilia melanogaster; identical to SEQ ID NO: 6

SEQ ID NO: 9 - protein, 37 aa, Drosophilia melanogaster; identical to SEQ ID NO: 6

SEQ ID NO: 10 - protein, 35 aa, Leptinotarsa decemlineata; identical to SEQ ID NO: 7

SEQ ID NO: 11 - DNA, 34 bp, Artificial
  Artificial = synthetic construct
ttttggatccaccatgaccaccatacactcgatc

SEQ ID NO: 12 - DNA, 31 bp, Artificial
  Artificial = synthetic construct
tttttctagactatgtcttcatgtcgacgtc

SEQ ID NO: 13 - DNA, 31 bp, Artificial
  Artificial = synthetic construct
ttttgaattctgctcgatttgcggggacaag

SEQ ID NO: 14 - DNA, 31 bp, Artificial
  Artificial = synthetic construct
ttttgaattcaagcgggaggcggttcaagaa

SEQ ID NO: 15 - DNA, 31 bp, Artificial
  Artificial = synthetic construct
ttttaagcttctaagtatccgactggttttc